(19) United States
(12) Patent Application Publication
(10) Pub. No.: US 2004/0023901 A1
(43) Pub. Date: Feb. 5, 2004

(54) Nucleoside 5'-monophosphate mimics and their prodrugs

(76) Inventors: Phillip D. Cook, Fallbrook, CA (US); Guangyi Wang, Carlsbad, CA (US); Thomas W. Bruice, Carlsbad, CA (US); Vasanthankumar Rajappan, Carlsbad, CA (US); Kandasamy Sakthivel, San Diego, CA (US); Kathleen D. Tucker, Escondido, CA (US); Jennifer L. Brooks, Encinitas, CA (US); Janet M. Leeds, Encinitas, CA (US); Maria Eugenia Ariza, San Marcos, CA (US); Patrick C. Fagan, Escondido, CA (US)

Correspondence Address: MORRISON & FOERSTER LLP, 3811 VALLEY CENTRE DRIVE, SUITE 500, SAN DIEGO, CA, 92130-2332, US

(21) Appl. No.: 10/376,949

(22) Filed: Feb. 28, 2003

Related U.S. Application Data
(60) Provisional application No. 60/361,177, filed on Feb. 28, 2002.

(51) Int. Cl. C07H 19/04; A61K 31/675; A61K 31/7056

(52) U.S. Cl. 514/43; 514/80; 536/26.1; 548/112

(57) ABSTRACT

The present invention relates to novel nucleoside 5′-monophosphate mimics, which contain novel nucleoside bases and phosphate moiety mimics optionally having sugar-modifications. The nucleotide mimics of the present invention, in a form of a pharmaceutically acceptable salt, a pharmaceutically acceptable prodrug, or a pharmaceutical formulation, are useful as antiviral, antimicrobial, anticancer, and immunomodulatory agents. The present invention provides a method for the treatment of viral infections, microbial infections, and proliferative disorders. The present invention also relates to pharmaceutical compositions comprising the compounds of the present invention optionally in combination with other pharmaceutically active agents.

This application asserts priority to U.S. provisional application Serial No. 60/361,177 filed Feb. 28, 2002, which is incorporated herein by reference in its entirety.

### FIELD OF INVENTION

The present invention relates to novel nucleoside 5′-monophosphate mimics, which contain novel nucleoside bases and phosphate moiety mimics optionally having sugar-modifications. The nucleotide mimics of the present invention, in a form of a pharmaceutically acceptable salt, a pharmaceutically acceptable prodrug, or a pharmaceutical formulation, are useful as antiviral, antimicrobial, anticancer, and immunomodulatory agents. The present invention provides a method for the treatment of viral infections, microbial infections, and proliferative disorders. The present invention also relates to pharmaceutical compositions comprising the compounds of the present invention optionally in combination with other pharmaceutically active agents.

### BACKGROUND OF THE INVENTION

Viral infections are a major threat to human health and account for many serious infectious diseases. Hepatitis C virus (HCV), a major cause of viral hepatitis, infected more than 200 million people worldwide. Current treatment for HCV infection is restricted to immunotherapy with interferon-α alone or in combination with ribavirin, a nucleoside analog. This treatment is effective in only about half the patients. Hepatitis B virus (HBV) has acutely infected almost a third of the world's human population, and about 5% of the infected are chronic carriers of the virus. Chronic HBV infection causes liver damage that frequently progresses to cirrhosis and/or liver cancer later in the life. Despite the availability and widespread use of effective vaccines and chemotherapy, the number of chronic carriers approaches 400 million worldwide.

Human immunodeficiency virus (HIV) causes progressive degeneration of the immune system, leading to the development of AIDS. A number of drugs have been clinically used, including HIV reverse transcriptase inhibitors and protease inhibitors. Currently, combination therapies are widely used for the treatment of AIDS in order to reduce the drug resistance. Despite the progress in the development of anti-HIV drugs, AIDS is still one of the leading epidemic diseases. Therefore, there is still an urgent need for new, more effective HCV, HBV, and HIV drugs. The treatments of viral infections caused by other viruses such as herpes simplex virus (HSV), cytomeglavirus (CMV), influenza viruses, West Nile virus, small pox, Epstein-Barr virus (EBV), varicella-zoster virus (VZV), and respiratory syncytial virus (RSV) also need better medicines.

Bacterial infections long have been the sources of many infectious diseases. The widespread use of antibiotics produces many new strains of life-threatening bacteria. Fungal infections are another type of infectious diseases, some of which also can be life-threatening. There is an increasing demand for the treatment of bacterial and fungal infections. Antimicrobial drugs based on new mechanisms of action are especially important.

Proliferative disorders are one of the major life-threatening diseases and have been intensively investigated for decades. Cancer now is the second leading cause of death in the United States, and over 500,000 people die annually from this proliferative disorder. All of the various cells types of the body can be transformed into benign or malignant tumor cells. Transformation of normal cells into cancer cells is a complex process and thus far is not fully understood. The treatment of cancer consists of surgery, radiation, and chemotherapy. While chemotherapy can be used to treat all types of cancer, surgery and radiation therapy are limited to certain cancer at certain sites of the body. There are a number of anticancer drugs widely used clinically. Among them are alkylating agent such as cisplatin, antimetabolites, such as 5-fluorouracil, and gemcitabine. Although surgery, radiation, and chemotherapies are available to treat cancer patients, there is no cure for cancer at the present time. Cancer research is still one of the most important tasks in medical and pharmaceutical organizations.

Nucleoside analogs have been used clinically for the treatment of viral infections and proliferative disorders. Most of the nucleoside drugs are classified as antimetabolites. After they enter cells, nucleoside analogs are successively phosphorylated to nucleoside 5′-monophosphates, 5′-diphosphates, and 5′-triphosphates. In most cases, nucleoside triphosphates, e.g., 3′-azido-3′-deoxythymidine (AZT, an anti-HIV drug) triphosphate and arabinofuranosylcytosine (cytarabine, an anticancer drug) triphosphate, are the chemical entities that inhibit DNA or RNA synthesis, either through a competitive inhibition of polymerases or through incorporation of modified nucleotides into DNA or RNA sequences. Nucleosides may act also as their diphosphate. For instance, 2′-deoxy-2′,2′-difluorocytidine (gemcitabine, an anticancer drug) 5′-diphosphate has been shown to inhibit human ribonucleotide reductase. Nucleoside drugs that function as their 5′-monophosphates are also known. For example, bredinin 5′-monophosphate is a potent inhibitor of human inosine monophosphate dehydrogenase (IMPDH) and is used clinically as an immunosuppressant in organ transplantation. Ribavirin 5′-monophosphate is also a potent inhibitor of IMPDH and plays an important role for the treatment of HCV. A number of other nucleoside 5′-monophsophates also showed potent inhibition of de novo biosynthesis of purine and pyrimidine nucleotides.

Nucleotide 5′-monophosphates are negatively charged chemical entities, which efficiently can not penetrate cell membrane. Therefore, intensive efforts have been made in search of biologically useful prodrugs (Wagner et al., Med. Res. Rev. 2000, 20, 417-451; Jones et al., Antiviral Res. 1995, 27, 1-17; Perigaud et al., Adv. in Antiviral Drug Des. 1995, 2, 147-172). It is hoped that nucleoside 5′-monophosphate prodrugs could bypass the first cellular phosphorylation steps by nucleoside kinases. Although the prodrugs of nucleotides bearing natural phosphates showed certain in vitro and in vivo activities, several major obstacles remain to be overcome. The most obvious barrier is the inherent instability of the natural phosphates to cellular nucleases. Nucleotide prodrugs can help deliver negatively-charged nucleotides into cells, but may not significantly increase their cellular stability. In addition, nucleotides bearing natural 5′-monophosphate released from their prodrugs, like the nucleoside 5′-monophospahte anabolized from nucleoside drugs in cells, may stay at three phosphorylation stages (mono-, di- and triphosphate), the undesired cellular interactions may result from nucleotides at undesired phosphorylation stages. Consequently, nucleotide prodrugs may cause adverse effects.

In order to stabilize nucleoside 5′-monophosphates, many efforts have been made to modify the monophosphate moiety. One type of nucleoside 5′-monophosphate mimics is the substitution of one phosphate oxygen with other heteroatoms or functions (Jasko et al., Nucleosides Nucleotides 1993, 12, 879-893; Jankowska et al., J. Org. Chem. 1998, 63, 8150-8156; Hampton et al., Biochemistry 1969, 8, 2303-2311; Casara et al., Bioorg. Med. Chem. Lett. 1992, 2, 145-148; Allen et al., J. Med. Chem. 1978, 21, 742-746; Phelps et al., J. Med. Chem. 1980, 23, 1229-1232). Among these phosphate mimics are 5′-O-alkylphosphate, 5′-O-arylphosphate, 5′-P-alkylphosphonate, 5′-P-arylphosphonate, 5-phosphoramidate, 5′-phosphorothioate, and 5′-P-boranophosphate. This type of modifications on phosphorus usually produces diastereomers due to the formation of the phosphorus chiral center. These phosphate mimics are generally more stable to cellular nucleases than natural phosphate.

Another type of nucleoside 5′-monophosphate mimics has modifications at the 5′-position of nucleosides. Among them are 5′-O-phosphonomethyl nucleosides (Holy et al., Collection Czechoslovak Chem. Commun 1982, 47, 3447-3463), nucleoside 5′-deoxy-5′-thio-5′-phosphorothioate (Zhang et al., Organic Lett. 2001, 3, 275-278), 5′-deoxynucleoside 5′-phosphonate (Raju et al., J. Med. Chem. 1989, 32, 1307-1313), and 5′-deoxy-5′-C-phosphonomethyl nucleosides (Garvey et al., Biochemistry 1998, 37, 9043-9051, Matulic-Adamic et al., J. Org. Chem. 1995, 60, 2563-2569). Nucleosides containing 5′-sulfonic acids and sulfonamide also have been reported (Mundill et al., J. Med Chem. 1981, 24, 474-477; Kristinsson et al., Tetrahedron 1994, 50, 6825-6838; Peterson et al., J. Med Chem. 1992, 35, 3991-4000), which can be considered as nucleoside 5′-monophosphate analogs.

In the de novo biosynthesis of purine nucleotides, imidazole nucleotides play important roles. However, the nucleoside 5′-monophosphate mimics containing five-membered heterocycle bases are seldom explored. Thus far, only three such nucleotide mimics have been reported, which are all based on ribavirin (1-β-D-ribofuranosyl-1,2,4-triazole-3-carboxamide). The three known nucleotide mimics are 5′-deoxy-5′-C-phosphonomethyl ribavirin (Furetes et al., J. Med. Chem. 1974, 17, 642-645), ribavirin 5′-phosphoramidate (Allen et al, J. Med. Chem. 1978, 21, 742-746), and ribavirin 5′-sulfamate (Smee D. F., Antiviral Activity of Ribavirin 5′-Sulfamate in Nucleotide Analogues as Antiviral Agents, Ed. Martin, J. ACS Symposium Series 401, American Chemical Society, Washington, D.C., 1989).

Other nucleotide mimics have also been reported, which disclosures describe certain nucleotide 5′-monophosphate mimics (Rosowsky et al., U.S. Pat. No. 5,132,414, July/1992; Rosowsky et al, WO 9838202, September/1998; Herrmann et al., WO 9316092, August/I 993; Bischofberger et al, U.S. Pat. No. 5,798,340, August/1998; Bischofberger et al, US 2001/0041794, November/2001).

According to the invention, nucleotide mimics can be very useful in the inhibition of the de novo nucleotide biosynthesis, leading to the treatment of viral infection, microbial infections, proliferative disorders, and immunosuppression.

### SUMMARY OF THE INVENTION

As can be seen from the above discussion, there is a need for effective and safe nucleoside and nucleotide drugs, which should possess a desired biological activity and do not need cellular activations. Such a drug requires enzymatically stable nucleotides that themselves are the inhibitors or ligands of desired biological targets as accomplished with the nucleotide mimics of the present invention. In the cases where the essential enzymes in nucleotide biosynthesis pathways are desired biological targets, most likely, the drugs would be the nonhydrolyzable 5′-monophosphate mimics of nucleoside analogs, which do not require any phosphorylation, but effectively inhibit the enzyme functions. It is equally important that the nucleotide mimics should not be the substrates of major nucleoside degradation enzymes. The base- and sugar-moieties of nucleosides and nucleotides can be metabolized in cells. For instance, adenine, cytosine and guanine nucleosides and nucleotides may be deaminated by corresponding deaminases. Nucleosides and nucleotides can be degraded to nucleobases and sugars by cellular nucleoside phosphorylase. Apparently, these degradations reduce the effectiveness of nucleoside and nucleotide drugs.

In order to overcome the unsatisfactory properties of current nucleoside and nucleotide drugs, certain new, unconventional approaches are taken for the discovery of a new generation of nucleoside and nucleotide drugs. One of the approaches to enhance the nuclease stability of nucleotides is to replace the natural phosphate moieties of nucleotides with phosphate mimics. In the case of the 5′-monophosphate moiety, the 5′-oxygen of a furanose sugar can be replaced by methylene, halogenated methylene, sulfur, imido or substituted imido groups; the 5′-methylene of the furanose sugar can be replaced by halogenated methylene, substituted methylene; and the phosphate oxygen atoms can be replaced by a variety of functional groups such as borano, sulfur, amino, alkoxy, and alkyl. In addition, the phosphate may be replaced with non-phosphorus moieties such as sulfamates and sulfonates. The resulting nucleotide mimics may no longer be the substrates of cellular nucleases. In order to enhance the stability of base- and sugar moieties, a variety of modifications may be introduced. Thus, appropriately modified nucleotides enzymatically are stable and potentially useful as biologically active chemical entities. The present invention relates to nucleoside 5′-monophosphate mimics useful for the treatment of viral infections, microbial infections, cancer, and other human diseases.

The present invention discloses novel nucleoside 5′-monophosphate mimics, their prodrugs and their biological uses.

In one aspect, the present invention provides azole nucleoside 5′-monophosphate mimics that contain a phosphate mimic stable to chemical and enzymatic hydrolysis.

In another aspect of the invention, the novel nucleoside mono-phosphates are converted into prodrugs to enhance drug absorption and/or drug delivery into cells.

Another aspect of the present invention provides novel nucleoside 5′-monophosphate mimics as a composition for therapeutic use for treatment of viral infections, microbial infections, and proliferative disorders and immunosuppression.

An additional aspect of the present invention provides a method for the treatment of viral infections, microbial infections, proliferative disorders, and immunosuppression comprising administrating an azole nucleoside 5′-monophosphate mimic of the present invention.

In one embodiment of the present invention, a nucleotide mimic is provided as shown by Formula (I):

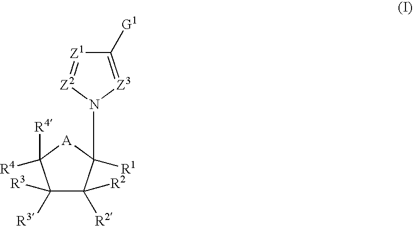

wherein A is O, S, CH₂, CHF, CF₂, or NH;

wherein R^{4′} is —L—R⁵ where L is selected from the group consisting of O, S, NH, NR, CH₂, CH₂O, CH₂S, CH₂NH, CH₂NR, CHY, CY₂, CH₂CH₂, CH₂CHY, and CH₂CY₂, where Y is F, Cl, Br, or selected from alkyl, alkenyl, and alkynyl optionally containing one or more heteroatoms;

wherein R⁵ is a moiety of Formula (II) or (III):

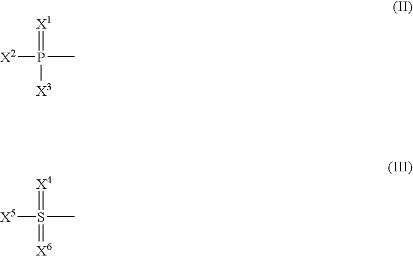

wherein X¹, X⁴, and X⁶ independently are O, S, NH, or NR;

wherein X², X³, and X⁵ are selected independently from the group consisting of H, F, OH, SH, NH₂, NHOH, N₃, CN, ⁻BH₃M⁺, R, OR, SR, NHR, NR₂ and R*, wherein R* is a prodrug substituent;

wherein R¹, R², R^{2′}, R³, R^{3′}, and R⁴ are selected independently from a group consisting of H, F, Cl, Br, I, OH, SH, NH₂, NHOH, N₃, NO₂, CHO, COOH, CN, CONH₂, COOR, R, OR, SR, SSR, NHR, and NR₂; alternatively, R² and R^{2′} together and R³ and R^{3′} together independently are ═O, ═S, or =J-Q, where J is N, CH, CF, CCl, or CBr, and Q is H, F, Cl, Br, N₃ or R;

wherein Z¹, Z², and Z³ are independently N, CH or C-G²;

wherein G¹ and G² are selected independently from a group consisting of H, F, Cl, Br, I, OH, SH, NH₂, NHOH, NHNH₂, N₃, NO, NO₂, CHO, COOH, CN, CONH₂, CONHR, C(S)NH₂, C(S)NHR, COOR, R, OR, SR, NHR, and NR₂;

wherein R is selected from the group consisting of alkyl, alkenyl, alkynyl, aryl, acyl, and aralkyl optionally containing one or more heteroatoms; and

with provisos that:

(1) at least one of X¹, X², and X³ is not O, OH or OR, when L is CH₂O which is linked to P through O;

(2) at least one of X¹, X², and X³ is not O, OH, OC₅H₆, or OCH₂C₅H₆, when L is CH₂CH₂, G¹ is CONH₂, Z¹ and Z³ are N, Z² is CH, R¹, R², R³, R⁴ are H, and R^{2′} and R^{3′} are OH;

(3) one of X² and X³ is not NH₂ when the other of X² and X³ is OH, X¹ is O, L is CH₂O which is linked to P through O, G¹ is CONH₂, CSNH₂, or CN, Z¹ and Z³ are N, Z² is CH, R¹, R², R³, and R⁴ are H, and R^{2′} and R^{3′} are OH;

(4) X⁵ is not NH₂ when X⁴ and X⁶ are O, L is CH₂O which is linked to S through O, G¹ is CONH₂, Z¹ and Z³ are N, Z² is CH, R¹, R², R³, and R⁴ are H, and R^{2′} and R^{3′} are OH;

(5) when L is CH₂O linked to P through CH₂ and R⁴ is alkyl, alkoxy, halomethyl, CH₂OH, CH₂N₃, CH₂CN, CH₂CH₂N₃, or CH₂CH₂OH, G¹ is not CONHR; and

(6) when L is CH₂CH₂, CH₂O, CH₂S, CH₂CHF, or CH₂CF₂ which is linked to P through CH₂ and R¹, R², R³, and R⁴ are hydrogen, G¹ is not CONHR.

In another embodiment of the present invention, a method is provided for the treatment of a viral infection comprising administering a therapeutically effective amount of a compound according to Formula (I), or a pharmaceutically acceptable salt or prodrug thereof.

In an additional embodiment of the present invention, a method is provided for the treatment of a proliferative disorder comprising administering a therapeutically effective amount of a compound according to Formula (I), or a pharmaceutically acceptable salt or prodrug thereof.

In a further embodiment of the present invention, a method is provided for the treatment of a microbial infection comprising administering a therapeutically effective amount of a compound according to Formula (1), or a pharmaceutically acceptable salt or prodrug thereof.

Furthermore, the present invention provides a method for immunomodulation comprising administering a therapeutically effective amount of a compound according to Formula (I), or a pharmaceutically acceptable salt or prodrug thereof.

In addition, the present invention provides a therapeutic composition comprising a therapeutically effective amount of a compound according to Formula (I), a pharmaceutically acceptable salt thereof, or a pharmaceutically acceptable prodrug thereof, optionally in combination with one or more active ingredients or a pharmaceutically acceptable carrier.

### DETAILED DESCRIPTION OF THE INVENTION

Preferred embodiments of the compound of the Invention of Formula (I) discussed above include:

a compound having Formula (IV):

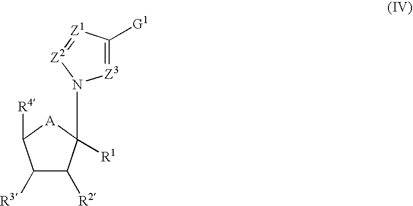

wherein R^{2′} and R^{3′} are independently H, F, or OH;

a compound having Formula (V):

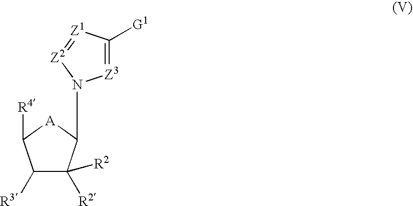

wherein R^{3′} is H, F, or OH;

a compound having Formula (VI):

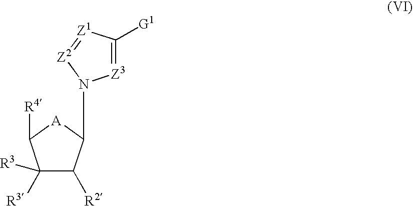

wherein R^{2′} is H, F, or OH;

a compound having Formula (VII):

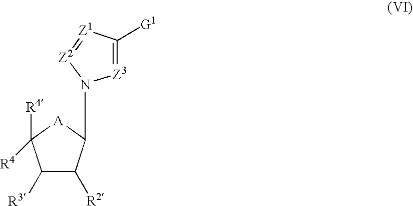

wherein R^{2′} and R^{3′} are independently H, F, or OH;

a compound having Formula (VIII):

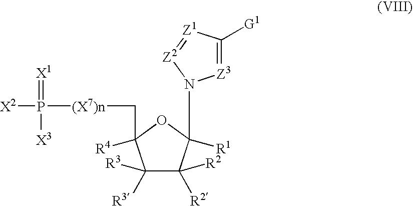

wherein X¹ is O or S;

wherein X² and X³ are selected independently from the group consisting of H, OH, SH, NH₂, F, NHOH, N₃, CN, ⁻BH₃M+, NHR, R, OR, SR, and R*, preferably wherein R* is 1,2-O-diacylglyceryloxy, 1,2-O-dialkylglyceryloxy, 1-O-alkyl-2-O-acylglyceryloxy, 1-O-acyl-2-O-alkylglyceryloxy, 1-S-alkyl-2-O-acyl-1-thioglyceryloxy, acyloxymethoxy, S-Lacyl-2-thioethoxy, S-pivaloyl-2-thioethoxy, acyloxymethoxy, pivaloyloxymethoxy, alkoxycarbonyloxymethoxy, or S-alkyldithio-S′-ethyoxy;

wherein X⁷ is O, S, NH, NMe, CH₂, CHF, CCl₂, or CF₂; and

wherein n is 0 or 1;

a compound having Formula (IX):

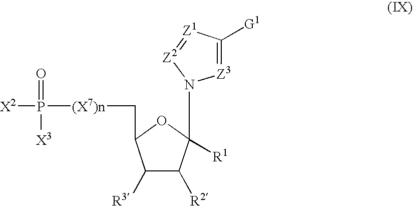

wherein X² and X³ are selected independently from the group consisting of H, F, OH, SH, NH₂, NHOH, N₃, CN, ⁻BH₃M+, NHR, R, OR, SR, and R*, preferably wherein R* is 1,2-O-diacylglyceryloxy, 1,2-O-dialkylglyceryloxy, 1-O-alkyl-2-O-acylglyceryloxy, 1-O-acyl-2-O-alkylglyceryloxy, 1-S-alkyl-2-O-acyl-1-thioglyceryloxy, acyloxymethoxy, S-acyl-2-thioethoxy, S-pivaloyl-2-thioethoxy, acyloxymethoxy, pivaloyloxymethoxy, alkoxycarbonyloxymethoxy, or S-alkyldithio-S′-ethyoxy;

wherein X⁷ is O, S, NH, NMe, CH₂, CHF, CCl₂, or CF₂;

wherein n is 0 or 1; and

wherein R^{2′} and R^{3′} are independently H, F, or OH;

a compound having Formula (X):

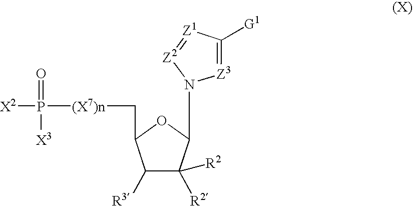

wherein X² and X³ are selected independently from the group consisting of H, F, OH, SH, NH₂, NHOH, N₃, CN, ⁻BH₃M+, NHR, R, OR, SR, and R*, preferably wherein R* is 1,2-O-diacylglyceryloxy, 1,2-O-dialkylglyceryloxy, 1-O-alkyl-2-O-acylglyceryloxy, 1-O-acyl-2-O-alkylglyceryloxy, 1-S-alkyl-2-O-acyl-1-thioglyceryloxy, acyloxymethoxy, S-acyl-2-thioethoxy, S-pivaloyl-2-thioethoxy, acyloxymethoxy, pivaloyloxymethoxy, alkoxycarbonyloxymethoxy, or S-alkyldithio-S′-ethyoxy;

wherein X⁷ is O, S, NH, NMe, CH₂, CHF, CCl₂, or CF₂;

wherein n is 0 or 1; and

wherein R^{3′} is H, F, or OH;

a compound having Formula (XI):

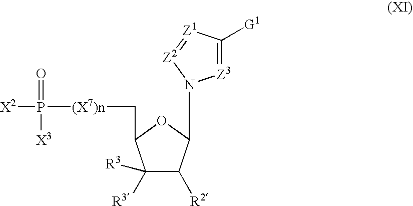

wherein X² and X³ are selected independently from the group consisting of H, F, OH, SH, NH₂, NHOH, N₃, CN, ⁻BH₃M⁺, NHR, R, OR, SR, and R*, preferably wherein R* is 1,2-O-diacylglyceryloxy, 1,2-O-dialkylglyceryloxy, 1-O-alkyl-2-O-acylglyceryloxy, 1-O-acyl-2-O-alkylglyceryloxy, 1-S-alkyl-2-O-acyl-1-thioglyceryloxy, acyloxymethoxy, S-acyl-2-thioethoxy, S-pivaloyl-2-thioethoxy, acyloxymethoxy, pivaloyloxymethoxy, alkoxycarbonyloxymethoxy, or S-alkyldithio-S′-ethyoxy;

wherein X⁷ is O, S, NH, NMe, CH₂, CHF, CCl₂, or CF₂;

wherein n is 0 or 1; and

wherein R^{2′} is H, F, or OH;

a compound having Formula (XII):

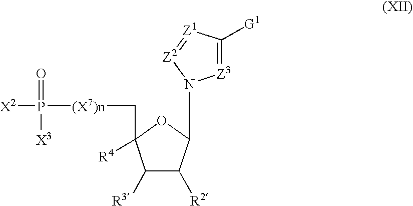

wherein X² and X³ are selected independently from the group consisting of H, OH, SH, F, OH, SH, NH₂, NHOH, N₃, CN, ⁻BH₃M⁺, NHR, R, OR, SR, and R*, preferably wherein R* is 1,2-O-diacylglyceryloxy, 1,2-O-dialkylglyceryloxy, 1-O-alkyl-2-O-acylglyceryloxy, 1-O-acyl-2-O-alkylglyceryloxy, 1-S-alkyl-2-O-acyl-1-thioglyceryloxy, acyloxymethoxy, S-acyl-2-thioethoxy, S-pivaloyl-2-thioethoxy, acyloxymethoxy, pivaloyloxymethoxy, alkoxycarbonyloxymethoxy, or S-alkyldithio-S′-ethyoxy;

wherein X⁷ is O, S, NH, NMe, CH₂, CHF, CCl₂, or CF₂;

wherein n is 0 or 1; and

wherein R^{2′} and R^{3′} are independently H, F, or OH;

a compound having Formula (XIII):

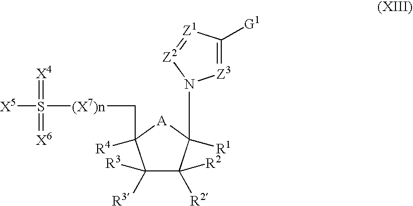

wherein X⁴ and X⁶ are independently O or S;

wherein X⁵ is selected from the group consisting of F, OH, SH, NH₂, NHOH, N₃, CN, ⁻BH₃M+, NHR, R, OR, SR, and R* preferably wherein R* is 1,2-O-diacylglyceryloxy, 1,2-O-dialkylglyceryloxy, 1-O-alkyl-2-O-acylglyceryloxy, 1-O-acyl-2-O-alkylglyceryloxy, 1-S-alkyl-2-O-acyl-1-thioglyceryloxy, acyloxymethoxy, S-acyl-2-thioethoxy, S-pivaloyl-2-thioethoxy, acyloxymethoxy, pivaloyloxymethoxy, alkoxycarbonyloxymethoxy, or S-alkyldithio-S′-ethyoxy;

wherein X⁷ is O, S, NH, NMe, CH₂, CHF, CCl₂, or CF₂; and

wherein n is 0 or 1;

a compound having Formula (XIV):

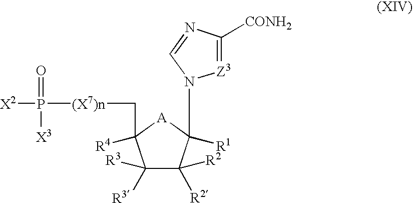

wherein X² and X³ are selected independently from the group consisting of F, OH, SH, NH₂, NHOH, N₃, CN, ⁻BH₃M⁺, NHR, R, OR, SR and R*, preferably wherein R* is 1,2-O-diacylglyceryloxy, 1,2-O-dialkylglyceryloxy, 1-O-alkyl-2-O-acylglyceryloxy, 1-O-acyl-2-O-alkylglyceryloxy, 1-S-alkyl-2-O-acyl-1-thioglyceryloxy, acyloxymethoxy, S-acyl-2-thioethoxy, S-pivaloyl-2-thioethoxy, acyloxymethoxy, pivaloyloxymethoxy, alkoxycarbonyloxymethoxy, or S-alkyldithio-S′-ethyoxy;

wherein X⁷ is O, S, NH, NMe, CH₂, CHF, CCl₂, or CF₂;

wherein n is 0 or 1; and

wherein Z³ is N, CH, C—OH, or C-ethynyl;

a compound having Formula (XV):

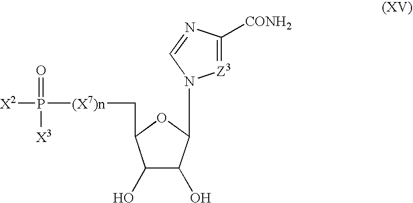

wherein X² and X³ are selected independently from the group consisting of F, OH, SH, NH₂, NHOH, N₃, CN, ⁻BH₃M⁺, NHR, R, OR, SR and R*, preferably wherein R* is 1,2-O-diacylglyceryloxy, 1,2-O-dialkylglyceryloxy, 1-O-alkyl-2-O-acylglyceryloxy, 1-O-acyl-2-O-alkylglyceryloxy, 1-S-alkyl-2-O-acyl-1-thioglyceryloxy, acyloxymethoxy, S-acyl-2-thioethoxy, S-pivaloyl-2-thioethoxy, acyloxymethoxy, pivaloyloxymethoxy, alkoxycarbonyloxymethoxy, or S-alkyldithio-S′-ethyoxy;

wherein Z³ is N, CH, C—OH, or C-ethynyl;

wherein X⁷ is O, S, NH, NMe, CH₂, CHF, CCl₂, or CF₂; and

wherein n is 0 or 1;

a compound having Formula (XVI):

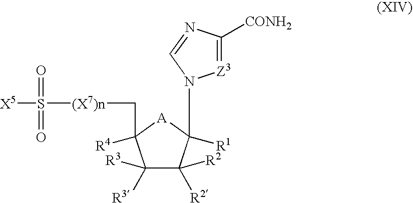

wherein X⁵ is selected from the group consisting of H, F, OH, SH, NH₂, NHOH, N₃, CN, ⁻BH₃M⁺, NHR, R, OR, SR, and R*, preferably wherein R* is 1,2-O-diacylglyceryloxy, 1,2-O-dialkylglyceryloxy, 1-O-alkyl-2-O-acylglyceryloxy, 1-O-acyl-2-O-alkylglyceryloxy, 1-S-alkyl-2-O-acyl-1-thioglyceryloxy, acyloxymethoxy, S-acyl-2-thioethoxy, S-pivaloyl-2-thioethoxy, acyloxymethoxy, pivaloyloxymethoxy, alkoxycarbonyloxymethoxy, or S-alkyldithio-S′-ethyoxy;

wherein X⁷ is O, S, NH, NMe, CH₂, CHF, CCl₂, or CF₂;

wherein n is 0 or 1; and

wherein Z³ is N, CH, C—OH, or C-ethynyl; or

a compound having Formula (XVII):

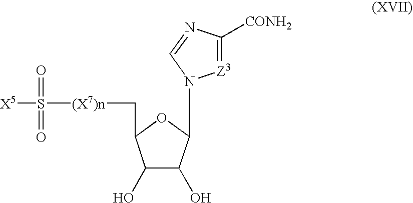

wherein X⁵ is selected from the group consisting of H, F, OH, SH, NH₂, NHOH, N₃, CN, ⁻BH₃M⁺, NHR, R, OR, SR, and R*, preferably wherein R* is 1,2-O-diacylglyceryloxy, 1,2-O-dialkylglyceryloxy, 1-O-alkyl-2-O-acylglyceryloxy, 1-O-acyl-2-O-alkylglyceryloxy, 1-S-alkyl-2-O-acyl-1-thioglyceryloxy, acyloxymethoxy, S-acyl-2-thioethoxy, S-pivaloyl-2-thioethoxy, acyloxymethoxy, pivaloyloxymethoxy, alkoxycarbonyloxymethoxy, or S-alkyldithio-S′-ethyoxy;

wherein X⁷ is O, S, NH, NMe, CH₂, CHF, CCl₂, or CF₂;

wherein n is 0 or 1; and

wherein Z³ is N, CH, C—OH, or C-ethynyl.

Any of the above compounds can be used in a pharmaceutical composition comprising therapeutically effective amount of any of the above-described compounds or a pharmaceutically acceptable salt thereof, or a pharmaceutically acceptable prodrug thereof. Such pharmaceutical compositions may also include one or more other biologically active agents. The pharmaceutical composition of the invention can be used for treatment of a viral infection, a microbial infection, a proliferative disorder, or for immunomodulation, or in related methods.

The definitions of certain terms and further descriptions of the above embodiments are given below.

The term moiety, unless otherwise specified, refers to a portion of a molecule. Moiety may be, but not limited to, a functional group, an acyclic chain, a phosphate mimic, an aromatic ring, a carbohydrate, a carbocyclic ring, or a heterocycle.

The term base, unless otherwise specified, refers to the base moiety of a nucleoside or nucleotide. The base moiety is the heterocycle portion of a nucleoside or nucleotide. The base moiety of a nucleotide mimic of Formula (I) is an azole heterocycle. The azole in the present invention refers to an imidazole, a 1,2,4-triazole, a 1,2,3-triazole, a pyrazole, a tetrazole, or a pyrrole, preferably imidazole or 1,2,4-triazole, i.e., wherein at least one of Z¹, Z² and Z³ is N. The azole heterocycle may contain one or more of the same or different substituents such as F, Cl, Br, I, OH, SH, NH₂, NHOH, N₃, NO₂, CHO, COOH, CN, CONH₂, COOR, R, OR, SR, SSR, NHR, and NR₂. Preferred substituents, include CONH₂, ethynyl, COOMe, OH, and most preferably CONH₂. In one preferred embodiment, one or two of Z¹, Z² and Z³ is N and at least one of Z¹, Z² and Z³ is CH. The nucleoside base is attached to the sugar moiety of the nucleotide mimic in such ways that both β-D- and β-L-nucleoside and nucleotide can be produced.

The term sugar refers to the ribofuranose portion of a nucleoside or a nucleotide.

The term modified sugar refers to a ribofuranose derivative or analog.

The sugar moiety of the invention refers to a ribofuranose, a ribofuranose derivative or a ribofuranose analog, as shown in Formula (I). The sugar moiety of nucleotide mimic of Formula (I) may contain one or more substituents at their C1-, C2-, C3-, C4, and C-5-position of the ribofuranose. Substituents may direct to either the α- or β-face of the ribofuranose. The nucleoside base that can be considered as a substituent at the C-1 position of the ribofuranose directs to the β-face of the sugar. The β-face is the side of a ribofuranose on which a purine or pyrimidine base of natural β-D-nucleosides is present. The α-face is the side of the sugar opposite to the β-face. A preferred embodiment of the sugar moiety is ribofuranose.

The term sugar-modified nucleoside refers to a nucleoside containing a modified sugar moiety.

The term nucleotide mimic, as used herein and unless otherwise specified, refers to an azole nucleoside 5′-monophosphate mimic.

The term phosphate mimic, unless otherwise specified, refers to a phosphate analog including, but not limited to, a phosphonate; phosphothioate, thiophosphate, P-boranophosphate, phosphoramidate, sulfamate, sulfonate, and sulfonamide. Preferred embodiments of the phosphate mimics include phosphonate, phosphorothioate, methylphosphonate, fluromethylphosphonate, difluoromethylphosphonate, vinylphosphonate, phenylphosphonate, sulfonate, fluorophosphate, dithiophosphorothioate, 5′-methylenephosphonate, 5′-difluoromethylenephosphonate, 5′-deoxyphosponate, 5′-aminophosphoramidate, and 5′-thiophosphate.

R⁵ is a phosphonate mimic:

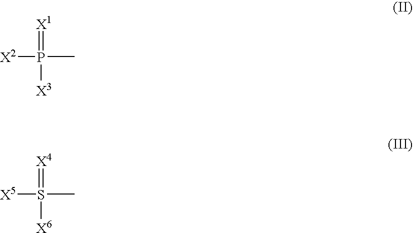

where X¹, X⁴, and X⁶ independently are O, S, NH, or NR; X², X³, and X⁵ are selected independently from the group consisting of H, F, OH, SH, NH₂, NHOH, N₃, CN, ⁻BH₃M⁺, R, OR, SR, NHR, and NR₂. The substituent ^{—}BH₃M⁺ is an ion pair, which is linked to phosphorus through the negatively charged boron. M⁺ is a cation.

The term cation, unless otherwise specified, refers to a positively charged ion, which is part of a nucleotide mimic of the invention. A pharmaceutical formulation contains a pharmaceutically acceptable cation, that is a cation that does not have or has a minimal adverse effect to a patient. A cation or pharmaceutically cation may be, but is not limited to, H⁺, Na⁺, K⁺, Li⁺, ½Ca⁺⁺, ½Mg⁺⁺, ammonium, alkylammonium, dialkylammonium, trialkylammonium or tertaalkylammonium.

R⁴⁻ of Formula (I) represents a combination (-L-R⁵) of a linker (L) and a phosphate mimic moiety (R⁵). L is either a one-atom, a two-atom, or a three-atom linker, which may, through either side, attach to the C4 position of the sugar moiety and the P or S of the phosphate mimic moiety. R⁵ represents a 5′-monophosphate mimic. X¹, X⁴, and X⁶ are double-bond compatible heteroatoms or groups; and X², X³, and X⁴ are each a univalent functional group which may replace the hydroxyls of a phosphate mimic as described above. Preferred embodiments for L include CH₂O, CH₂OCH₂, CH₂S, CH₂SCH₂, CH₂NHCH₂, CH₂, and CH₂CF₂.

The term alkyl, unless otherwise specified, refers to a saturated straight, branched, or cyclic hydrocarbon of C1 to C18. Alkyls may include, but not limited to, methyl, ethyl, n-propyl, isopropyl, cyclopropyl, n-butyl, isobutyl, t-butyl, cyclobutyl, n-pentyl, isopentyl, neopentyl, cyclopentyl, n-hexyl, cyclohexyl, dodecyl, tetradecyl, hexadecyl, and octadecyl.

The term alkenyl, unless otherwise specified, refers to an unsaturated hydrocarbon of C2 to C18 that contains at least one carbon-carbon double bond and may be straight, branched or cyclic. Alkenyls may include, but not limited to, olefinic, propenyl, allyl, 1-butenyl, 3-butenyl, 1-pentenyl, 4-pentenyl, 1-hexenyl, and cyclohexenyl.

The term alkynyl, unless otherwise specified, refers to an unsaturated hydrocarbon of C2 to C18 that contains at least one carbon-carbon triple bond and may be straight, branched or cyclic. Alkynyls may include, but not limited to, ethynyl, 1-propynyl, 2-propynyl, 1-butynyl, and 3-butynyl.

The term aryl, unless otherwise specified, refers to an aromatic moiety with or without one or more heteroatom. Aryls may include, but are not limited to, phenyl, biphenyl, naphthyl, pyridinyl, pyrrolyl, and imidazolyl optionally containing one or more substituents. The substituents may include, but are not limited, hydroxy, amino, thio, halogen, cyano, nitro, alkoxy, alkylamino, alkylthio, hydroxycarbonyl, alkoxycarbonyl, and carbamoyl.

The term aralkyl, unless otherwise specified, refers to a moiety that contains both an aryl and an alkyl, an alkenyl, or an alkynyl. Aralkyls can be attached through either the aromatic portion or the non-aromatic position. Aralkyls may include, but are not limited to, benzyl, phenylethyl, phenylpropyl, methylphenyl, ethylphenyl, propylphenyl, butylphenyl, phenylethenyl, phenylpropenyl, phenylethynyl, and phenylpropynyl.

The term acyl, unless otherwise specified, refers to alkylcarbonyl. Acyls may include, but are not limited to, formyl, acetyl, fluoroacetyl, difluoroacetyl, trifluoroacetyl, chloroacetyl, dichloroacetyl, trichloroacetyl, propionyl, benzoyl, toluoyl, butyryl, isobutyryl, and pivaloyl.

The term heteroatom refers to oxygen, sulfur, nitrogen, or halogen. When one or more heteroatoms are attached to alkyl, alkenyl, alkynyl, acyl, aryl, or arakyl, a new functional group may be produced. For instance, when one or more heteroatoms are attached to an alkyl, substituted alkyls may be produced, including, but not limited to, fluoroalkyl, chloroalkyl, bromoalkyl, iodoalkyl, alkoxy, hydroxyalkyl, alkylamino, aminoalkyl, alkylthio, thioalkyl, azidoalkyl, cyanoalkyl, nitroalkyl, carbamoylalkyl, carboxylalkyl, acylalkyl, acylthioethoxy, acyloxymethoxy, 1,2-O-diacylglyceryloxy, 1,2-O-dialkylglyceryloxy, and 1-O-alkyl-2-O-acylglyceryloxy.

The term halogen or halo refers to fluorine, chlorine, bromine, or iodine.

The term function refers to a substituent. Functions may include, but not limited to, hydroxy, amino, sulfhydryl, azido, cyano, halo, nitro, hydroxycarbonyl, alkoxycarbonyl, or carboxyl either protected or unprotected.

R of Formula (I) is a univalent substituent and present on the base, sugar and phosphate mimic moieties. R is selected from the group consisting of alkyl, alkenyl, alkynyl, aryl, acyl, and aralkyl optionally containing one or more heteroatoms, which are as defined above. Preferred R groups include OH, O-benyzl, and O-benzoyl. Preferred R groups on the phosphate mimic moiety include CH₃, CH₂F, vinyl, phenyl, CHF₂, and CH₂CH₃.

R* is a prodrug substituent. The term prodrug, unless otherwise specified, refers to a masked (protected) form of a nucleotide mimic of Formula (1) that is formed when one or more of X², X³ or X⁵ is R*. The prodrug of a nucleoside 5′-monophosphate mimic can mask the negative charges of the phosphate mimic moiety entirely or partially, or mask a heteroatom substituted alkyl, aryl or aryalkyl (W, see below) attached to a phosphate or phosphate mimic moiety in order to enhance drug absorption and/or drug delivery into cells. The prodrug can be activated either by cellular enzymes such as lipases, esterases, reductases, oxidases, nucleases or by chemical cleavage such as hydrolysis to release (liberate) the nucleotide mimic after the prodrug enters cells. Prodrugs are often referred to as cleavable prodrugs. Prodrugs substituents include, but are not limited to: proteins; antibiotics (and antibiotic fragments); D- and L-amino acids attached to a phosphate moiety or a phosphate mimic moiety via a carbon atom (phosphonates), a nitrogen atom (phosphoamidates), or an oxygen atom (phosphoesters); peptides (up to 10 amino acids) attached to a phosphate moiety or a phosphate mimic moiety via a carbon atom (phosphonates), a nitrogen atom (phosphoamidates), or an oxygen atom (phosphoesters); drug moieties attached to a phosphate moiety or a phosphate mimic moiety via a carbon atom (phosphonates), a nitrogen atom (phosphoamidates), or an oxygen atom (phosphoesters); steroids; cholesterols; folic acids; vitamins; polyamines; carbohydrates; polyethylene glycols (PEGs); cyclosaligenyls; substituted 4 to 8-membered rings, with or without heteroatom substitutions, with 1,3-phosphodiester, 1,3-phosphoamidate/phosphoester or 1,3-phosphoamidate attachments or phosphate mimic moiety; acylthioethoxy, (SATE) RCOSCH₂CH₂O—; RCOSCH₂CH₂O—W—O—; RCOSCH₂CH₂O—W—S—; RCOSCH₂CH₂O—W—NH—; RCOSCH₂CH₂O—W—; RCOSCH₂CH₂O—W—CY₂—; acyloxymethoxy, RCOOCH₂O—; RCOOCH₂O—W—O—; RCOOCH₂O—W—S—; RCOOCH₂O—W—NH—; RCOOCH₂O—W—; RCOOCH₂O—W—CY₂—; alkoxycarbonyloxymethoxy, ROCOOCH₂O—; ROCOOCH₂O—W—O—; ROCOOCH₂O—W—S—; ROCOOCH₂O—W—NH—; ROCOOCH₂O—W—; ROCOOCH₂O—W—CY₂—; acylthioethyldithioethoxy (DTE) RCOSCH₂CH₂SSCH₂CH₂O—; RCOSCH₂CH₂SSCH₂CH₂O—W—; RCOSCH₂CH₂SSCH₂CH₂O—W—O—; RCOSCH₂CH₂SSCH₂CH₂O—W—S—; RCOSCH₂CH₂SSCH₂CH₂O—W—NH—; RCOSCH₂CH₂SSCH₂CH₂O-CY₂—; acyloxymethylphenylmethoxy (PAOB) RCO₂—C₆H₄—CH₂—O—; RCO₂—C₆H₄—CH₂—O—W—; RCO₂—C₆H₄—CH₂—O—W—O—; RCO₂—C₆H₄—CH₂—O—W—S—; RCO₂—C₆H₄—CH₂—O—W—NH—; RCO₂—C₆H₄—CH₂—O—W-CY₂—; 1,2-O-diacyl-glyceryloxy, RCOO—CH₂—CH(OCOR)—CH₂O—; 1,2-O-dialkyl-glyceryloxy, RO—CH₂—CH(OR)—CH₂O—; 1,2-S-dialkyl-glyceryloxy, RS—CH₂—CH(SR)—CH₂O—; 1-O-alkyl-2-O-acyl-glyceryloxy, RO—CH₂—CH(OCOR)—CH₂O—; 1-S-alkyl-2-O-acyl-glyceryloxy, RS—CH₂—CH(OCOR)—CH₂O—, 1-O-acyl-2-O-alky-glyceryloxy, RCOO—CH₂—CH(OR)—CH₂O—; 1-O-acyl-2-S-alky-kglyceryloxy, RCOO—CH₂—CH(SR)—CH₂O—; any substituent attached via a, carbon, nitrogen or oxygen atom to a nucleoside di- or tri-phosphate mimic that liberates the di- or tri-phosphate mimic in vivo.

A combination of prodrug substituents may be attached (conjugated) to one or more X², X³ and X⁵ positions on a nucleoside mono-phosphate mimic. W is alkyl, aryl, aralkyl as described above or a heterocycle. Preferred prodrug substituents (R*) in positions X², X³ or X⁵ include 2,3-O-diacylglyceryloxy, 2,3-O-dialkylglyceryloxy, 1-O-alkyl-2-O-acylglyceryloxy, 1-O-acyl-2-O-alkylglyceryloxy, 1-S-alkyl-2-O-acyl-1-thioglyceryloxy, acyloxymethoxy, S-acyl-2-thioethoxy, S-pivaloyl-2-thioethoxy, acyloxymethoxy, pivaloyloxymethoxy, alkoxycarbonyloxymethoxy, S-alkyldithio-S′-ethyoxy acyloxymethoxy, S-acyl-2-thioethoxy, S-pivaloyl-2-thioethoxy, pivaloyloxymethoxy, alkoxycarbonyloxymethoxy, and S-alkyldithio-S′-ethyoxy.

The term microbial infection refer to an infection caused by a bacteria, parasite, virus or fungus. Examples of microbes that cause such infections include: Acanthamoeba, African Sleeping Sickness (Trypanosomiasis), amebiasis, American Trypanosomiasis (Chagas Disease), Bilharzia (Schistosomiasis), cryptosporidiosis (diarrheal disease, Cryptosporidium Parvum), Giardiasis (diarrheal disease, Giardia lamblia), hepatitis A, B, C, D, E, leishmaniasis (skin sores and visceral), malaria (Plasmodium falciparum), Salmonella enteritides infection (stomach cramps, diarrhea and fever), tuberculosis (mycobacterium tuberculosis), varicella (chicken pox), yellow fever, pneumonias, urinary tract infections (Chlamydia and Mycoplasma), meningitis & meningococcal septicemia, skin and soft tissue infections (Staphylococcus aureus), lower respiratory tract infections (bacterial pathogens or hepatitis C).

Common infections caused by microbes are further outlined in the following chart:

| Infection | Bacteria | Fungus | Protozoa | Virus |
|---|---|---|---|---|
| AIDS | | | | X |
| Athlete's Foot | | X | | |
| Chicken Pox | | | | X |
| Common Cold | | | | X |
| Diarrheal Disease | X | | X | X |
| Flu | | | | X |
| Genital Herpes | | | | X |
| Malaria | X | | X | |
| Meningitis | X | | | |
| Pneumonia | X | X | | |
| Sinusitis | X | X | | |
| Skin Disease | X | X | X | X |
| Strep Throat | X | | | |
| Tuberculosis | X | | | |
| Urinary Tract | X | | | |
| Infections | | | | |
| Vaginal Infections | X | X | | |
| Viral Hepatitis | | | | X |

The term pharmaceutically acceptable carrier refers to a pharmaceutical formulation which serves as a carrier to deliver negatively-charged nucleotide mimics of the present invention into cells. Liposome, polyethylenimine, and cationic lipids are the examples of those carriers.

The term “treat” as in “to treat a disease” is intended to include any means of treating a disease in a mammal, including (1) preventing the disease, i.e., avoiding any clinical symptoms of the disease, (2) inhibiting the disease, that is, arresting the development or progression of clinical symptoms, and/or (3) relieving the disease, i.e., causing regression of clinical symptoms.

A. Synthesis of Nucleotide Mimics

The synthesis of the nucleotide mimics of the present invention are conducted either through traditional organic synthesis or through parallel organic synthesis, either in solution-phase or on solid supports. The nucleotide mimics are characterized using Mass and NMR spectrometry.

Nucleosides for the Preparation of Nucleotide Mimics

The novel nucleosides that are used to prepare the nucleotide mimics of the present invention can be synthesized either according to published, known procedures or can be prepared using well-established synthetic methodologies (Chemistry of Nucleosides and Nucleotides Vol. 1, 2, 3, edited by Townsend, Plenum Press, 1988, 1991, 1994); Handbook of Nucleoside Synthesis by Vorbrüggen Ruh-Pohlenz, John Wiley & Sons, Inc., 2001; The Organic Chemistry of Nucleic Acids by Yoshihisa Mizuno, Elsevier, 1986). The nucleosides can be converted to their corresponding nucleotide mimics by established phosphorylation methodologies.

One of the general approaches for the preparation of novel nucleosides is as follow: 1. properly protected, modified sugars including 1-, 2-, 3-, 4-, 5-substituted furanose derivatives and analogs which are not commercially available need to be synthesized; 2. The modified sugars are condensed with properly substituted azole heterocycles to yield modified nucleosides; 3. The resulting nucleosides can be further derivatised at nucleoside level through reactions on the base and/or sugar moieties. For maximal efficiency, the nucleosides may be prepared through solution or solid-phase parallel synthesis.

Prior publications reported a variety of ribofuranose analogs including ribofuranose derivatives, cyclopentyl derivatives, thioribofuranose derivatives, and azaribofuranose derivatives, which, with appropriate protection and substitution, can be used for the condensations with nucleoside bases. Well-established procedures and methodologies in the literature can be used for the preparation of the modified sugar used in the present invention (Sanhvi et al., Carbohydrate Modifications in Antisense Research, ACS symposium Series, No. 580, American Chemical Society, Washington, D.C.). A large number of 2-, and 3-substituted ribofuranose analogs are well documented and can be readily synthesized accordingly (Hattori et al., J. Med. Chem. 1996, 39, 5005-5011; Girardet et al., J. Med. Chem. 2000, 43, 3704-3713)). A number of 4-, and 5′-substitued sugars have also been reported and the procedures and the methodologies are useful for the preparation of the modified sugars used in the invention (Gunic et al., Bioorg. Med. Chem. 2000, 9, 163-170; Wang et al., Tetrahedron Lett. 1997, 38, 2393-2396). Methodologies for the preparation of 4-thiosugars and 4-azasugars are also available (Rassu et al., J. Med. Chem. 1997, 40, 168-180; Leydier et al, Nucleosides Nucleotides 1994, 13, 2035-2050). Cyclopentyl carbocyclic sugars have been used widely to prepare carbocyclic nucleoside and the preparative procedures are also well documented (Marquez, In Advances in Antiviral Drug Design; De Clercq, E. Ed.; JAI press Inc. Vol. 2, 1996; pp89-146). These methodologies can be applied readily in the preparation of azole nucleosides.

The favorable nucleoside bases of the present invention are triazole derivatives, imidazole derivatives, pyrazole derivatives, pyrrole derivatives, and tetrazole derivatives. The azole heterocycles bearing a variety of substituents are well known compounds and can be readily synthesized according to known procedures. A number of imidazole and triazole analogs as nucleoside bases have been well documented (Chemistry of Nucleosides and Nucleotides Vol. 3, edited by Townsend, Plenum Press, 1994). The condensations of sugars with nucleoside bases to yield nucleosides are the most frequently used reactions in nucleoside chemistry. Well-established procedures and methodologies can be found in the literature (Vorbruggen et al., Chem. Ber. 1981, 114, 1234-1268, 1279-1286; Wilson et al., Synthesis, 1995, 1465-1479). There are several types of standard condensation reactions widely used, including: 1. trimethylsilyl triflate-catalyzed coupling reaction between 1-O-acetylribofuranose derivatives and silylated nucleoside bases, often used for the preparation of ribonucleosides; 2. tin chloride-catalyzed coupling reactions between 1-O-methyl or 1-O-acetylribofuranose derivatives and silylated nucleoside bases, often used to prepare 2′-deoxyribonucleosides; 3. SN2 type substitutions of 1-halosugar by nucleoside bases in the presence of a base such as sodium hydride for the preparation of both ribonucleosides and 2′-deoxyribonucleosides; and 4. Less often used, but still useful, fusion reactions between sugars and nucleoside bases without solvent.

Modifications can be done at nucleoside level. The sugar moieties of synthesized nucleosides can be further derivatised. There are a variety types of reactions which can be used to modify the sugar moiety of nucleosides. The reactions frequently used include deoxygenation, oxidation/addition, substitution, and halogenation. The deoxygenations are useful for the preparation of 2′-deoxy-, 3′-deoxy, and 2′,3′-dideoxynucleosides. A widely-used reagent is phenyl chlorothionoformate, which reacts with the hydroxy of nucleosides to yield a thionocarbonate. The treatment of the thionocarbonate with tributyltin hydride and AIBN yields deoxygenated nucleosides. The oxidation/addition includes the conversion of a hydroxy group to a carbonyl group, followed by a nucleophilic addition, resulting in C-alkylated nucleosides and C-substituted nucleosides. The substitution may be just a simple replacement of a hydroxyl proton by alkyl, or may be a conversion of a hydroxyl to a leaving group, followed by a nucleophilic substitution. The leaving group is usually a halogen, mesylate, tosylate, nisylate, or a triflate. A variety of nucleophiles can be used, resulting in nucleosides are 2-, or 3-substituted nucleosides. The halogenation can be used to prepare 1′-halo, 2′-halo, 3′-halo-, 4′-halonucleosides. Chlorination and fluorination are commonly used and result in important fluoro-sugar and chloro-sugar nucleosides.

The Preparation of Nucleoside 5′-Monophosphate Mimics

Nucleoside 5′-phosphorothioate can be synthesized from the reaction of nucleoside with thiophosphoryl chloride in the presence of 1,8-bis(dimethylamino)naphthalene (proton sponge) in anhydrous pyridine (Fisher et al., J. Med. Chem. 1999, 42, 3636). For example, 1-(β-D-ribofuranosyl)-1,2,4-triazole-3-carboxamide 5′-phosphorothioate (1) and 5-ethynyl-1-(β-D-ribofuranosylimidazole-4-carboxamide 5′-phosphorothioate (2) were prepared through this reaction.

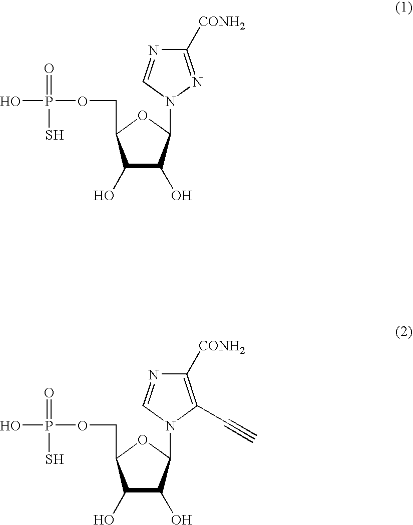

Nucleoside 5′-P-alkylphosphonates can be prepared from the reaction of a nucleoside with alkylphosphonic acid in the presence of dicyclohexylcarbodiimide (DCC). For example, 1-(2,3-O-isopropylidene-1-β-D-ribofuranosyl-1,2,4-triazole-3-carboxamide (3) prepared according to a reported procedure (Kini et al., J. Med. Chem., 1990, 33, 44-48) was reacted with methylphosphonic acid in the presence of DCC in anhydrous pyridine to yield methyl phosphonate derivative (4). The deprotection using Dowex-H+resin in methanol yielded 1-(5-O-methylphosphonyl-β-D-ribofuranosyl)-1,2,4-triazole-3-carboxamide (5).

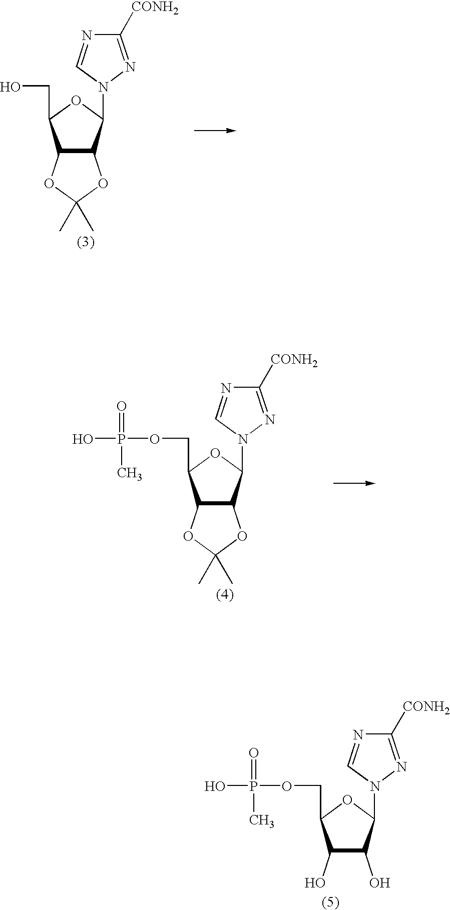

Similarly, the reactions of compound 3 with fluoromethylenephosphonic acid (Hamilton et al., J. Chem. Soc., Perkin. Trans. 1, 1999, 1051-1056) and difluoromethylphosphonic acid (prepared by treating commercially available diethyl difluoromethylphosphonate with bromotrimethylsilane in methylene chloride) in the presence of DCC, followed by deprotection with Dowex-H⁺, yielded compound (6) and (7), respectively. Compounds (8)-(12) were also prepared through this type of reactions.

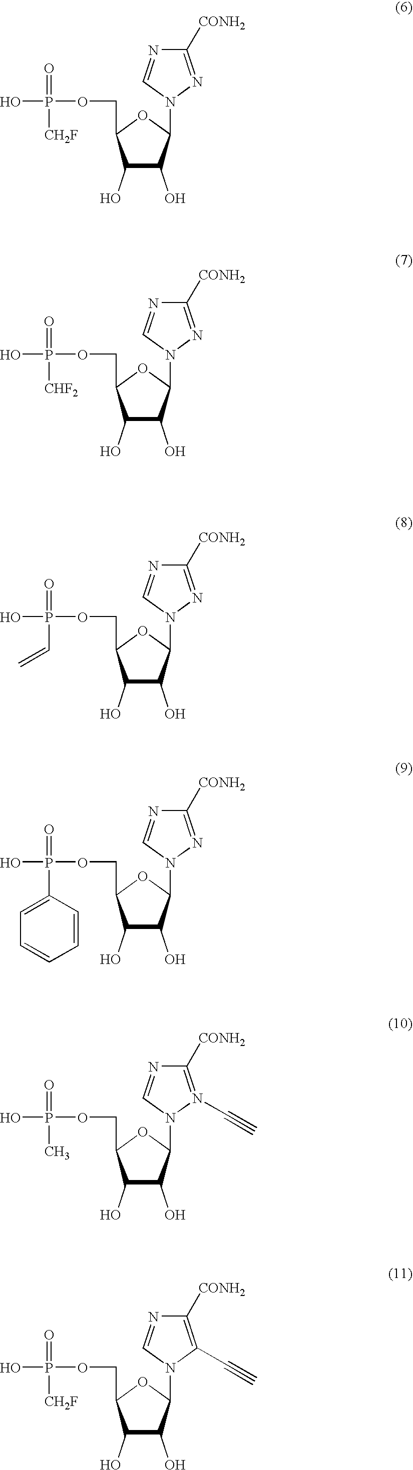

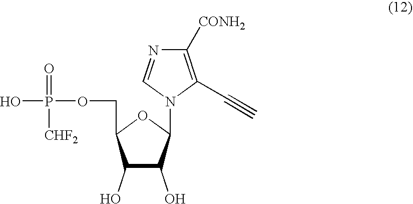

Compound (13) (Kini et al., J. Med. Chem. 1990, 33, 44-48) was reacted with (diethoxyphosphinyl)methyl triflate (Xu et al., J. Org. Chem. 1996, 61, 7697-7701) in the presence of sodium hydride and the resulting product (14) was treated with bromotrimethylsilane, followed by hydrolysis with Dowex-H⁺ in methanol, yielded the phosphonate (15).

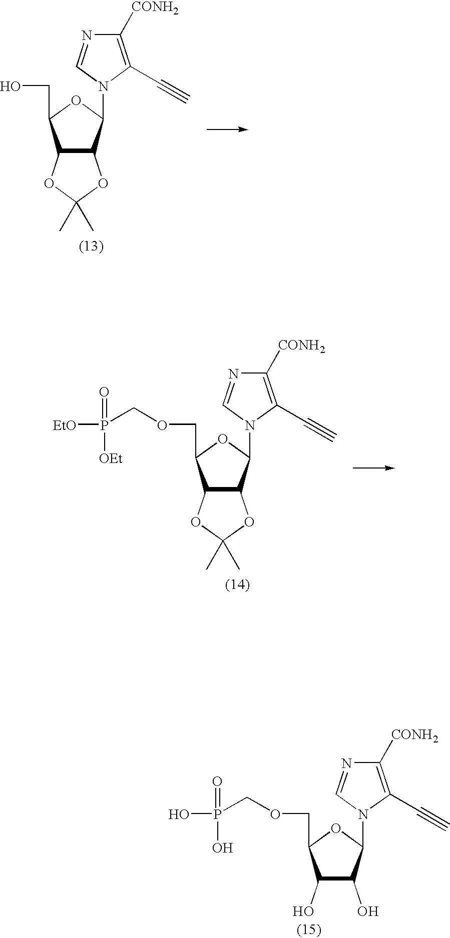

1-(5-O-Phosphonylmethyl-β-D-ribofuranosyl)-1,2,4-triazole-3-carboxamide (19) was also prepared by a slightly different procedure. Methyl 1-(2,3,5-tri-O-acetyl-β-D-ribofuranosyl)-1,2,4-triazole-3-carboxylate (16) was reacted with sodium methoxide in methanol, followed by treatment with dimethoxypropane, perchloric acid in acetone. The resulting (17) was reacted with (diethoxyphosphinyl)methyl triflate in the presence of sodium hydride to yield compound (18). Deprotection of (18) with methanolic ammonia, followed by treatment with bromotrimethysilane and then with Dowex-H⁺, yielded compound (19).

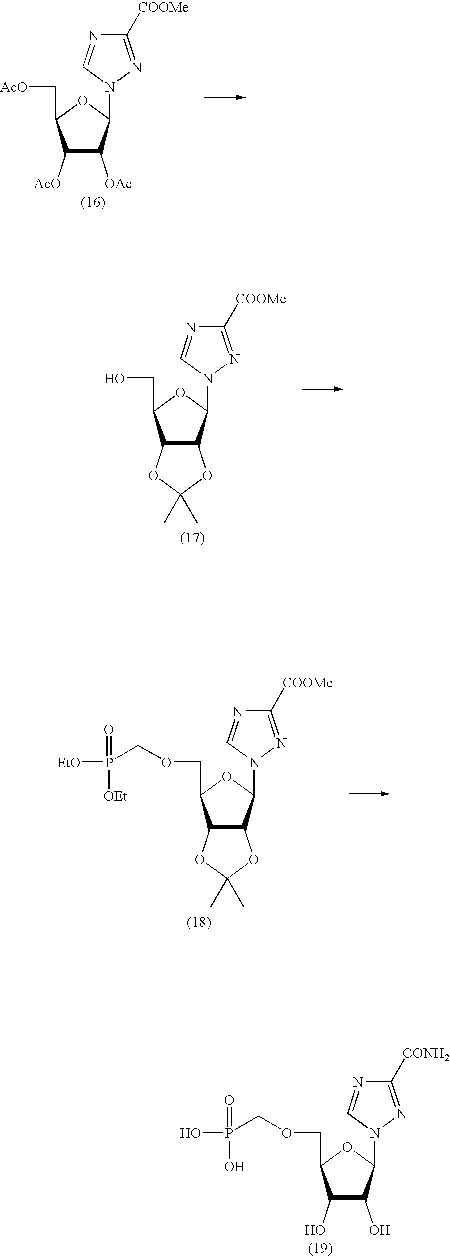

Compound (3) was reacted with thiolacetic acid under Mitsunobu reaction condition using triphenylphosphine and diisopropyl azodicarboxylate to yield the S-ester (21). After removal of the acetyl group under oxygen-free condition, the resulting (22) was reacted with methylphosphonic acid in the presence of DCC, followed by treatment with DOWEX 50WX8-100 resin in methanol, to yield the methylphosphonate (23). By another procedure, compound (24) was prepared from the reaction of (22) with (di-O-ethyl)phosphonomethyl trifluoromethanesulfonate and subsequent deprotection.

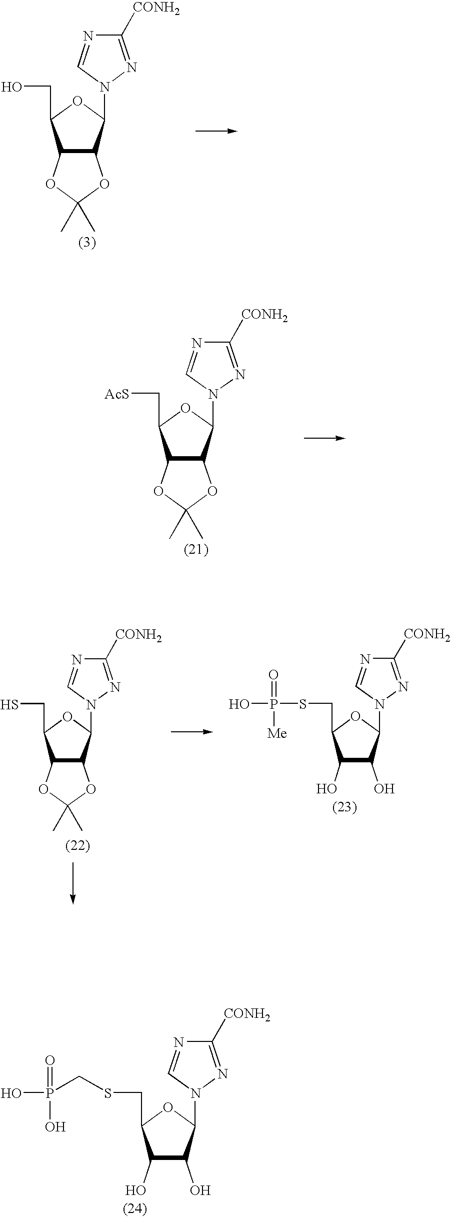

The reaction of 1-β-D-ribofuranosyl-1,2,4-triazole-3-carboxamide (25) with iodine in the presence of triphenylphosphine yielded (26), which was refluxed with excess sodium sulfite to give the 5′-sulfonic acid (27). The reaction of (26) with sodium dithiophosphate in water yielded the dithio compound (28).

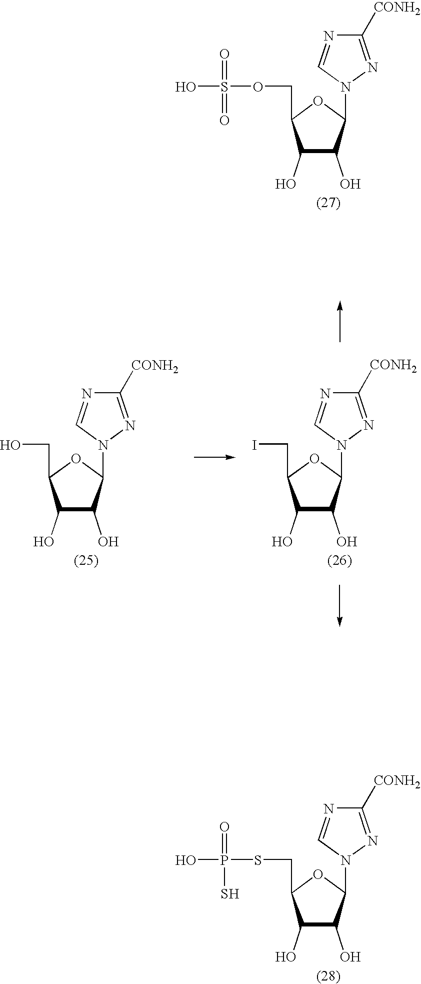

Compound (26) was reacted with sodium azide to give the azido compound (29), which was converted to the amine (30) by hydrogenolysis over palladium. The reaction of (30) with O-diethylphosphonomethyl trifluoromethanesulfonate yielded (31), which was subjected to deprotection with bromotrimethylsilane to give the 5′-phosphonylmethylamino compound (32).

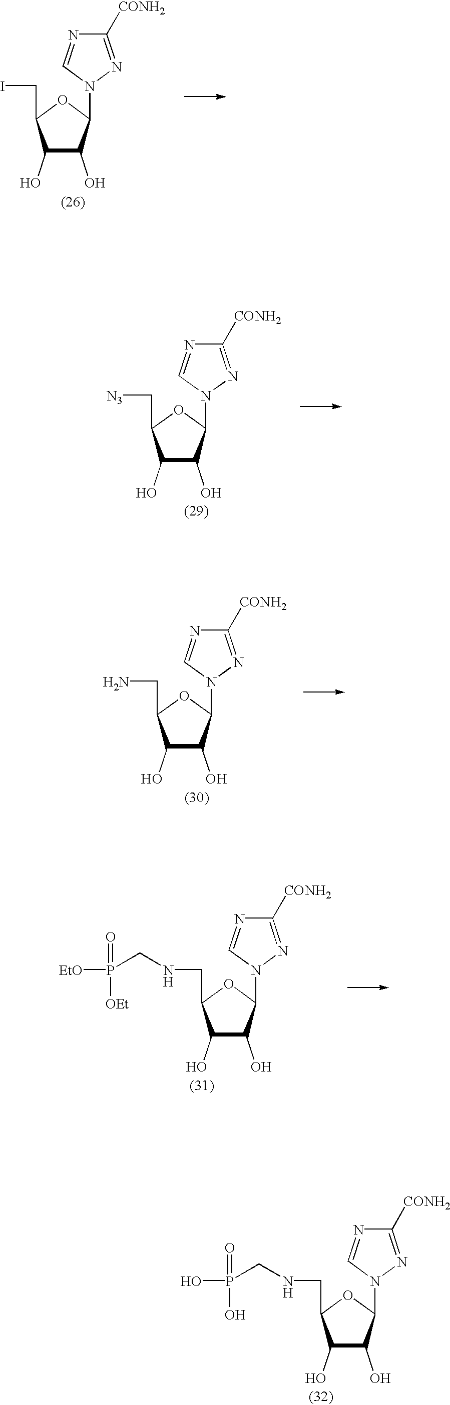

Compound (25) was treated with tert-butyldimethylsilylchloride in pyridine and then further reacted with benzoyl chloride. The TBDMS group of the resulting intermediate was removed with tetrabutylammonium fluoride in THF to yield compound (33). The reaction of (33) with fluorophosphonic acid in presence of DCC in pyridine and the resulting product (34) was subjected to a deprotection with aqueous ammonia to yield the fluorophosphonate (35). Similarly, the reaction of compound (30) with diphenylhydrogen phosphonate, followed by protection with aqueous ammonia, yielded 1-(5-O-hydrogenphosphonyl-β-D-ribofuranosyl-1,2,4-triazole-3-carboxamide (36).

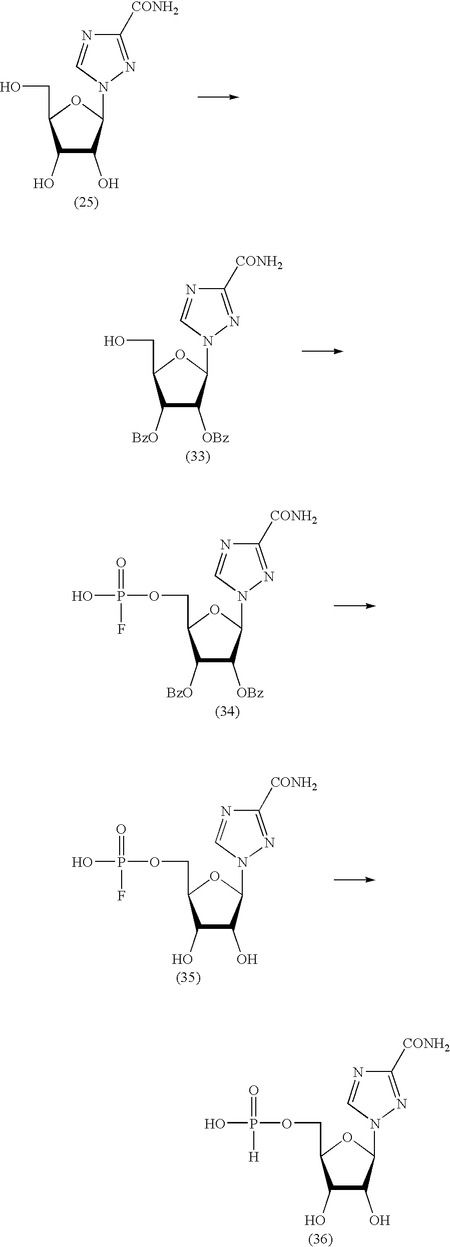

Compound (26) was benzoylated and the resulting (37) was reacted with triethylphosphite at 100° C. to yield the phosphonate analog (38). Treatment of (38) with bromotrimethylsilane, followed by deprotection with aqueous ammonia, yielded 1-[5-deoxy-5-(phosphonyl)-β-D-ribofuranosyl]-1,2,4-triazole-3-carboxamide (39). Similarly, the reaction of (37) with bis(trimethylsilyl) phosphite, followed by deprotection with aqueous ammonia, yielded 1-[5-(deoxy-5-hydroxyl-H-phosphinyl)-β-D-ribofuranosyl)-1,2,4-triazole-3-carboxamide (40).

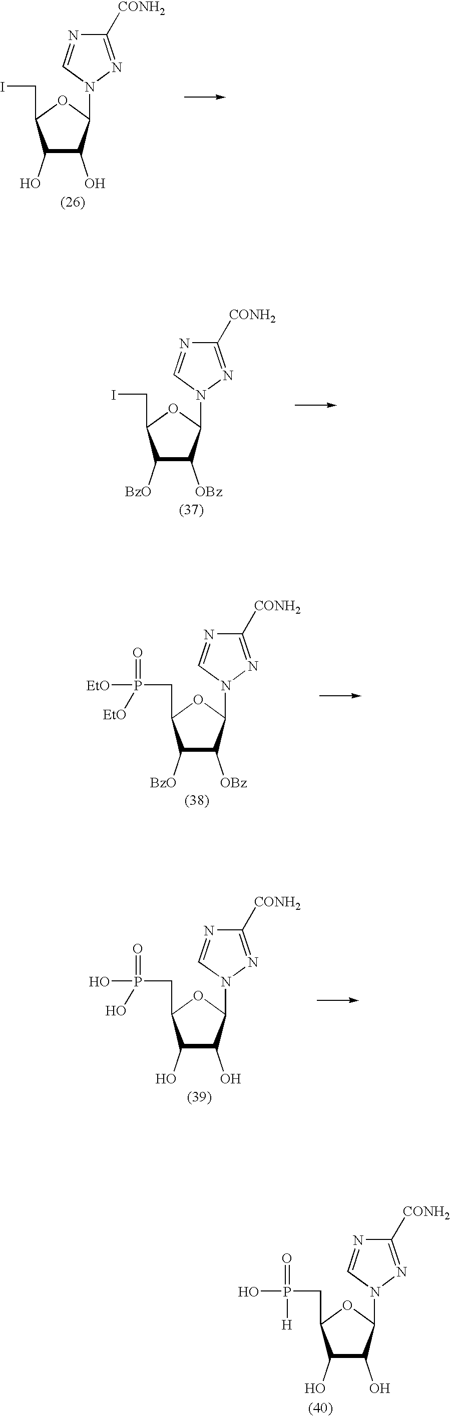

Compound (44) was also synthesized, but starting from the carbohydrate (41), which was prepared according to a similar procedure as published (Raju et al., J. Med. Chem. 1989, 32, 1307-1313). Compound (42) was prepared according to a reported procedure (Schipper et al. J. Am. Chem. Soc; 1952, 74, 350-353). The condensation of (41) and the silylated form of (42) in the presence of stannic chloride yielded the nucleotide (43), which was treated with bromotrimethylsilane, followed by deprotection with methanolic ammonia, to give compound (44).

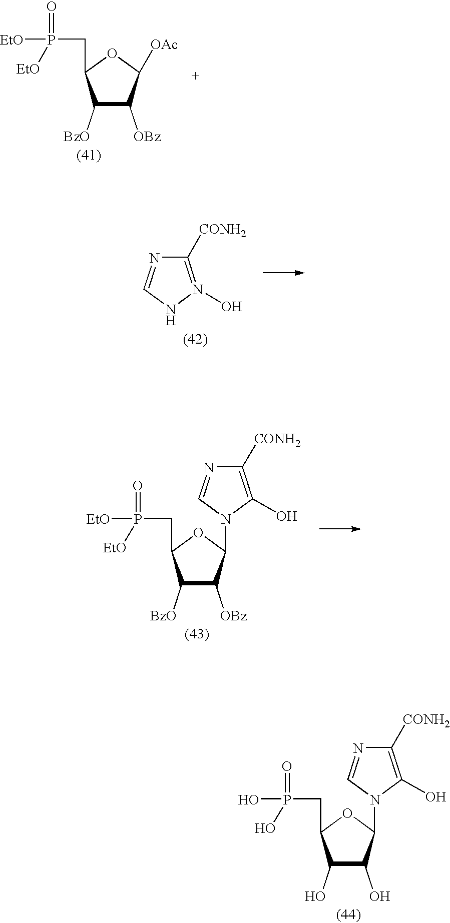

Compound (45) was prepared according to a published procedure (Matulic-Adamic et al., J. Org. Chem; 1995, 60, 2563-60). Compound (46) was refluxed with hexamethyldisilazane to obtain a silylated derivative. The condensation of (45) and the trimethylsilylated derivative of (46) in the presence of stannic chloride in acetonitrile yielded the difluoromethylene phophonate ester (47), which was treated with methanolic ammonia, followed by removal of benzyl and ethyl group, to give 1-(5-deoxy-5-phosphonyldifluoromethylene)-β-D-ribofuranosyl)-1,2,4-triazole-3-carboxamide (48).

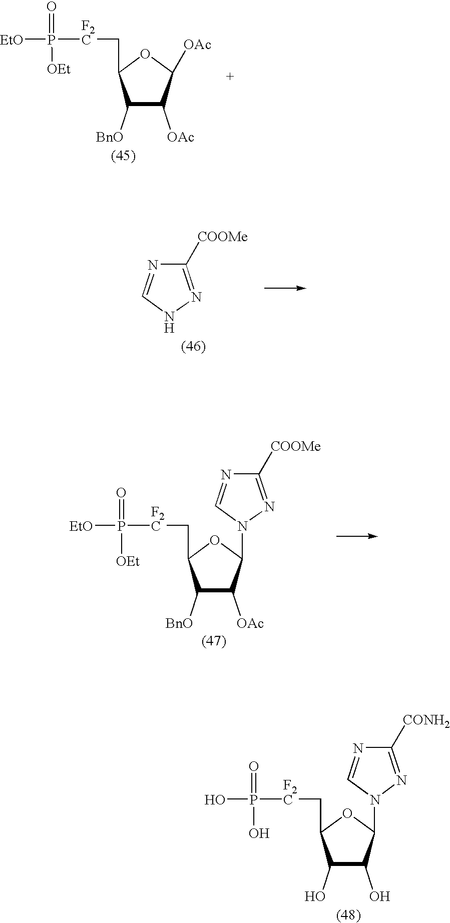

Compound (42) was refluxed with hexamethyldisilazane to obtain a silylated derivative of (42). The condensation of (45) and the silylated derivative of (42) in the presence of titanium (IV) chloride in nitromethane yielded the nucleotide (49), which was treated with boron chloride, followed by treatment with bromotrimethylsilane, to yield 4-carbamoyl-1-[5,6-dideoxy-6-(dihydroxyphosphinyl)-6,6-difluoro-p-D-ribofuranosyl]-1,3-imidazolium-5-olate (50).

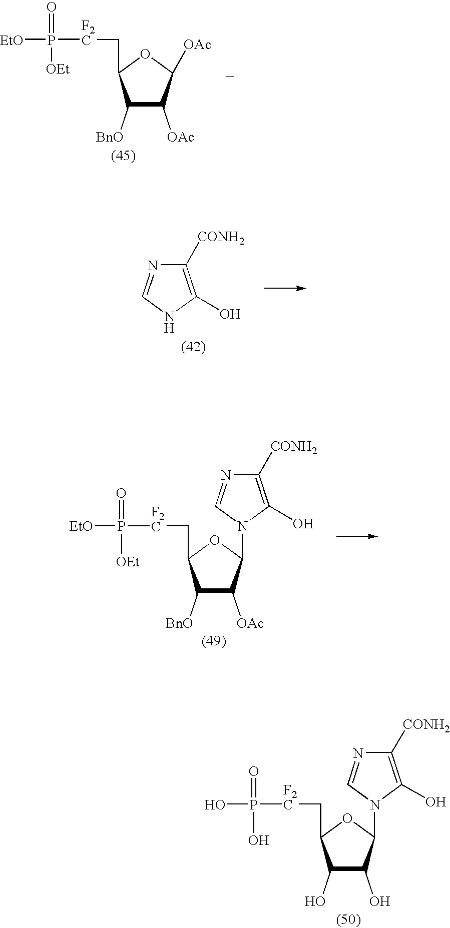

9-Fluorinemethyl H-phosphonothioate (51), prepared according to a reported procedure (Jankowska et al, Tetrahedron Letters; 1997, 38, 2007-2010), was reacted with (33) in presence of trimethyacetyl chloride to yield compound (52). The deprotection with aqueous methylamine afforded 1-(5-hydrogen-P-thiophosponyl-β-D-ribofuranosyl-1,2,4-triazole-3-carboxamide (53). When (52) was reacted with sulfur in lutidine/methylene choloride and subsequent treatment with 0.1 N sodium hydroxide yielded 1-β-D-ribofuranosyl-1,2,4-triazole-3-carboxamide 5′-dithiophosphorothioate (54).

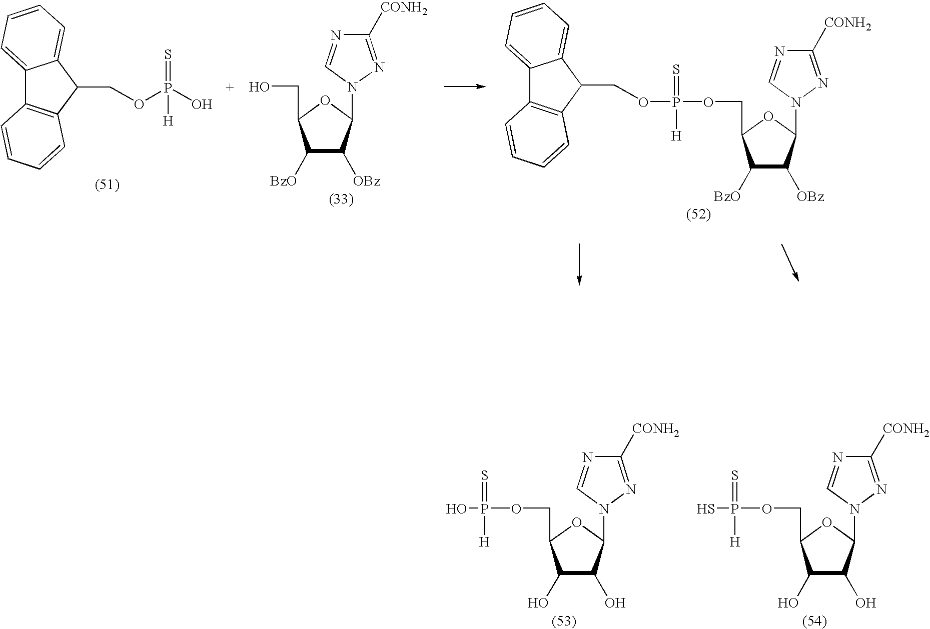

Prodrug approach is one of the efficient methods to deliver polar, negatively-charged nucleotide mimics into cells. A number of prodrug approaches for nucleoside 5′-monophosphates have been developed and potentially can be applied to the nucleotide mimics of the present invention. The nucleotide mimic prodrugs may include, but are not limited to, alkyl phosphate esters, aryl phosphate ester, acylthioethyl phosphate esters, acyloxymethyl phosphate esters, 1,2-O-diacylglyceryl phosphate esters, 1,2-O-dialkylglyceryl phosphate esters, and phosphoramidate esters. These masking groups can be readily attached to the nucleoside mimics of the present invention. The resulting compounds can serve as the prodrugs of the nucleotide mimics. For example, the treatment of compound (4) with S-pivaloyl-2-thioethanol in the presence of 1-(mesitylene-2-sulfonyl)-3-nitro-1,2,4-triazole, followed by a deprotection of isopropylidene, yielded compound (56), a prodrug of compound (5).

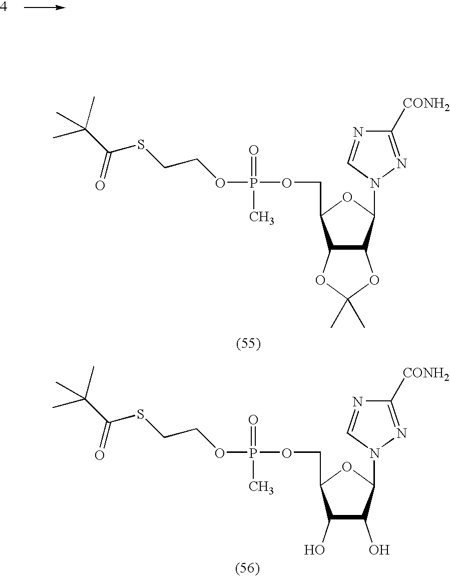

Compound (57) was a minor product (19%) from the reaction of compound (3) with methylphosphonic acid in the presence of DCC. After removal of isopropylidene, the resulting (58) was treated with tri-n-butylstannyl methoxide, followed by reaction with iodomethyl pivalate in the presence of tetra-n-butylammonium bromide, to give compound (59), another prodrug of compound (5).

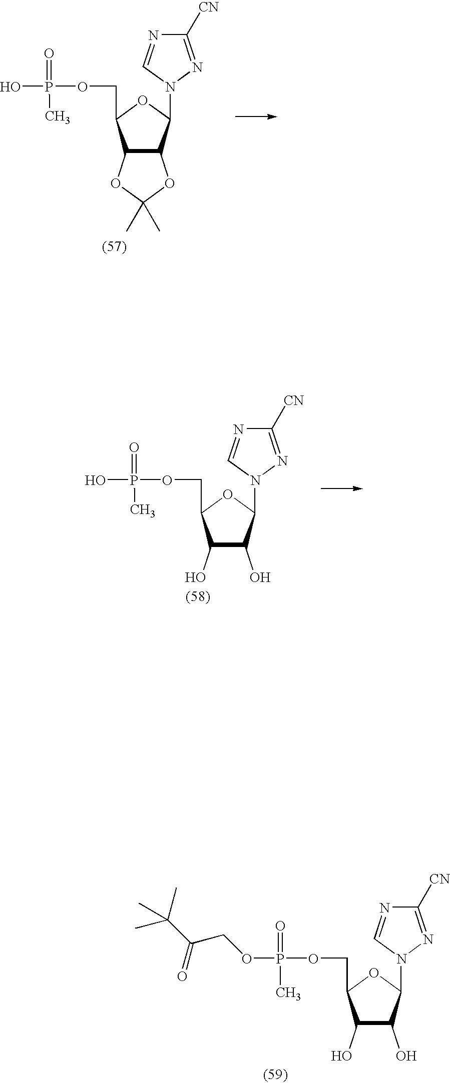

B. Biological Applications and Administration

The nucleoside 5′-monophosphate mimics of the present invention are useful for the inhibition of a variety of enzymes including, but not limited to, inosine monophosphate dehydrogenases (IMPDH), orotidine monophosphate decarboxylases, AICAR transformylases, guanosine monophosphate synthetases, adenylosuccinate synthetases and adenylosuccinate lyases, thymidylate synthases, and protein kinases.

The nucleoside 5′-monophosphate mimics of the present invention are useful as human therapeutics for the treatment of infectious diseases caused by viruses including, but not limited to, HIV, HBV, HCV, hepatitis delta virus (HDV), HSV, CMV, small pox, West Nile virus, influenza viruses, measles, rhinovirus, RSV, VZV, EBV, vaccinia virus, and papilloma virus.

The nucleoside 5′-monophosphate mimics of the present invention are useful for the treatment of one or more infectious diseases caused by bacteria and fungus.

The nucleoside 5′-monophosphate mimics that have potent cytotoxicities to fast-dividing cancerous cells are useful for the treatment of proliferative disorders, including, but not limited to, lung cancer, liver cancer, prostate cancer, colon cancer, breast cancer, ovary cancer, melanoma, and leukemia.

The nucleoside 5′-monophosphate mimics of the present invention are useful as immunomodulatory agents, especially as immuosuppressants.

In order to overcome drug resistance, combination therapies are widely used in the treatment of infectious diseases and proliferative disorders. The nucleotide mimics or their prodrugs of the present invention may be therapeutically administered as a single drug, or alternatively may be administered in combination with one or more other active chemical entities to form a combination therapy. The other active chemical entities may be a small molecule, a polypeptide, or a polynucleotide.

The pharmaceutical composition of the present invention comprises at least one of the compounds represented by Formula (I) or pharmaceutically acceptable salts or prodrugs thereof as active ingredients. The compositions include those suitable for oral, topical, intravenous, subcutaneous, nasal, ocular, pulmonary, and rectal administration. The compounds of the invention can be administered to mammalian individuals, including humans, as therapeutic agents. For example, the compounds of the invention are useful as antiviral agents. The present invention provides a method for the treatment of a patient afflicted with a viral infection comprising administering to the patient a therapeutically effective antiviral amount of a compound of the invention.

The term “viral infection” as used herein refers to an abnormal state or condition characterized by viral transformation of cells, viral replication and proliferation. Viral infections for which treatment with a compound of the invention will be particularly useful include the virues mentioned above.

A “therapeutically effective amount” of a compound of the invention refers to an amount which is effective, upon single or multiple dose administration to the patient, in controlling e.g., the growth of the virus, bacteria or fungus or controlling cell proliferation or in prolonging the survivability of the patient beyond that expected in the absence of such treatment. As used herein, “controlling the growth” e.g., of the virus, bacteria or fungui or proliferating cells refers to slowing, interrupting, arresting or stopping e.g., the viral, bacteria or fungal or abnormal proliferation or transformation of cells or abnormal proliferation or the replication and proliferation of the virus, bacteria or fungus and does not necessarily indicate a total elimination of the virus, bacteria or fungus or proliferating cells.

Accordingly, the present invention includes pharmaceutical compositions comprising, as an active ingredient, at least one of the compounds of the invention in association with a pharmaceutical carrier. The compounds of this invention can be administered by oral, parenteral (intramuscular, intraperitoneal, intravenous (IV) or subcutaneous injection), topical, transdermal (either passively or using iontophoresis or electroporation), transmucosal (e.g., nasal, vaginal, rectal, or sublingual) or pulmonary (e.g., via dry powder inhalation) routes of administration or using bioerodible inserts and can be formulated in dosage forms appropriate for each route of administration.

Solid dosage forms for oral administration include capsules, tablets, pills, powders, and granules. In such solid dosage forms, the active compound is admixed with at least one inert pharmaceutically acceptable carrier such as sucrose, lactose, or starch. Such dosage forms can also comprise, as is normal practice, additional substances other than inert diluents, e.g., lubricating, agents such as magnesium stearate. In the case of capsules, tablets, and pills, the dosage forms may also comprise buffering agents. Tablets and pills can additionally be prepared with enteric coatings.

Liquid dosage forms for oral administration include pharmaceutically acceptable emulsions, solutions, suspensions, syrups, with the elixirs containing inert diluents commonly used in the art, such as water. Besides such inert diluents, compositions can also include adjuvants, such as wetting agents, emulsifying and suspending agents, and sweetening, flavoring, and perfuming agents.

Preparations according to this invention for parenteral administration include sterile aqueous and non-aqueous solutions, suspensions, or emulsions. Examples of non-aqueous solvents or vehicles are propylene glycol polyethylene glycol, vegetable oils, such as olive oil and corn oil, gelatin, and injectable organic esters such as ethyl oleate. Such dosage forms may also contain adjuvants such as preserving, wetting, emulsifying, and dispersing agents. They may be sterilized by, for example, filtration through a bacteria retaining filter, by incorporating sterilizing agents into the compositions, by irradiating the compositions, or by heating the compositions. They can also be manufactured using sterile water, or some other sterile injectable medium, immediately before use.

Compositions for rectal or vaginal administration are preferably suppositories which may contain, in addition to the active substance, excipients such as cocoa butter or a suppository wax. Compositions for nasal or sublingual administration are also prepared with standard excipients well known in the art.

Topical formulations will generally comprise ointments, creams, lotions, gels or solutions. Ointments will contain a conventional ointment base selected from the four recognized classes: oleaginous bases; emulsifiable bases; emulsion bases; and water-soluble bases. Lotions are preparations to be applied to the skin or mucosal surface without friction, and are typically liquid or semiliquid preparations in which solid particles, including the active agent, are present in a water or alcohol base. Lotions are usually suspensions of solids, and preferably, for the present purpose, comprise a liquid oily emulsion of the oil-in-water type. Creams, as known in the art, are viscous liquid or semisolid emulsions, either oil-in-water or water-in-oil. Topical formulations may also be in the form of a gel, i.e., a semisolid, suspension-type system, or in the form of a solution.

Finally, formulations of these drugs in dry powder form for delivery by a dry powder inhaler offer yet another means of administration. This overcomes many of the disadvantages of the oral and intravenous routes.

The dosage of active ingredient in the compositions of this invention may be varied; however, it is necessary that the amount of the active ingredient shall be such that a suitable dosage form is obtained. The selected dosage depends upon the desired therapeutic effect, on the route of administration, and on the duration of the treatment desired. Generally, dosage levels of between 0.001 to 10 mg/kg of body weight daily are administered to mammals.

The following examples are put forth so as to provide those of ordinary skill in the art with a complete disclosure and description of how to prepare and use the compounds disclosed and claimed herein. Efforts have been made to ensure accuracy with respect to numbers (e.g., amounts, temperature, etc.) but some errors and deviations may remain.

### EXAMPLES

A. Chemical Synthesis

The following examples for the preparation of the nucleotide mimics of the present invention are given in this section. The examples herein are not intended to limit the scope of the limitation to the present invention in any way. The nucleotide mimics of the present invention can be prepared by those skilled in the art of nucleoside and nucleotide chemistry.

### Example 1

### 1-β-D-ribofuranosyl-1,2,4-triazole-3-carboxamide 5′-phosphothioate (1)

To a suspension of 1-β-D-ribofuranosyl-1,2,4-triazole-3-carboxamide (122 mg. 0.5 mmol) in 2.5 mL of anhydrous pyridine was added proton sponge® [1,8-bis(dimethylamino)naphthalene] (107 mg. 0.5 mmol) at 0-5° C. under argon atmosphere, followed by addition of thiophosphoryl chloride (0.1 mL, 1 mmol). The mixture was stirred at this temperature for 30 minutes and then quenched with 3 mL of 1 M triethylammonium bicarbonate buffer. The pyridine and proton sponge® were extracted into chloroform by shaking with 2 mL of chloroform, and the aqueous layer was subjected to HPLC purification on C₁₈ column. Collected fractions were lyophilized to give 60 mg of the titled compound (1).

### Example 2

### 5-Ethynyl-1-(1-D-ribofuranosyl)imidazole-4-carboxamide 5′-phosphorothioate (2)

To a suspension of 50 mg (0.187 mmol) of 5-ethynyl-1-β-D-ribofuranosylimidazole-4-carboxamide (EICAR) in 1.2 mL of anhydrous pyridine was added 115.5 mg 90.54 mmol) proton sponge® at 0-5° C. under argon atmosphere. To this mixture was added, drop-wise, thiophosphoryl chloride (63 mg, 38 μL, 0.37 mmol). The mixture was stirred at this temperature for 30 minutes and then quenched with 3 mL of 1M triethylammonium bicarbonate buffer. The pyridine and proton sponge® were extracted into chloroform by shaking with 2 ml of chloroform and the aqueous layer was subjected for purification on reverse-phase HPLC. The material was purified on C₁₈ column and then lyophilized to get 16.7 mg of titled compound (2).

### Example 3

### 1-(5-O-Methylphosphonyl-β-D-ribofuranosyl-1,2,4-triazole-3-carboxamide (5)

2′,3′-O-Isopropylidene-1-β-D-ribofuranosyl-1,2,4-triazole-3-carboxamide (142 mg, 0.5 mmol), synthesized according to a reported procedure (Kini et al., J. Med. Chem; 1990, 33, 44-48), was co-evaporated with anhydrous pyridine (3×5 mL) under reduced pressure and taken into 5 mL of anhydrous pyridine. To the above solution under argon atmosphere were added dicyclohexylcarbodiimide (206 mg, 1.0 mmol) and methyl phosphonic acid (58 mg, 0.6 mmol). The mixture was stirred at 38° C. for 36 hours. Water (5 mL) was added to the mixture after cooling to room temperature. The dicyclohexylurea precipitated was filtered off and the filtrate was concentrated under reduced pressure and then filtered again. After evaporation, the concentrate was co-evaporated with toluene to remove traces of pyridine.

The crude product (105 mg) was dissolved in methanol (5 mL) and Dowex 50W×8-100 resin (1 g, pre-washed with water and methanol), was added. The mixture was heated at 50° C. for 2 hours, filtered through a short pad of cotton in a small tube, and then the resin was thoroughly washed with water. The filtrate was concentrated to yield a viscous residue which was purified on C₁₈ column. The fractions collected was lyophilized to give 34 mg of the titled compound (5).

### Example 4

### 1-[5-O-(Fluoromethyl)phosphonyl-β-D-ribofuranosyl]-1,2,4-triazole-3-carboxamide (6)

1-(2,3-O-Isopropylidene-β-D-ribofuranosyl)-1,2,4-triazole-3-carboxamide (142 mg, 0.5 mmol) and (fluoromethyl)phosphonic acid (60 mg, 0.6 mmol) prepared according to a reported procedure (Hamilton et al., J. Chem. Soc; Perkin. Trans. 1, 1999, 1051-1056) were co-evaporated with anhydrous pyridine (3×5 mL) under reduced pressure and taken into 3 mL of anhydrous pyridine. To the above solution under argon atmosphere was added DCC (202 mg. 1.0 mmol) and the resulting mixture was stirred at 38° C. for 24 hours. Water (3 mL) was added to the mixture after cooling it to room temperature, and the resulting dicyclohexylurea was filtered off. The filtrate was concentrated under reduced pressure and again filtered. After evaporation of the remaining solvent, the concentrate was co-evaporated with toluene to remove traces of pyridine.

The crude product (80 mg) was dissolved in 5 mL of methanol and Dowex 50W×8-100 resin (1 g) was added. The mixture was heated at 50° C. for 2 hours, filtered, washed thoroughly with water. The filtrate was concentrated to give a viscous residue which was purified on C₁₈ column. The fractions collected was lyophilized to give 25 mg of titled compound (6).

### Example 5

### 1-[5-O-(Difluoromethyl)phosphonyl-β-D-ribofuranosyl]-1,2,4-triazole-3-carboxamide (7)

1-(2,3-O-Isopropylidene-β-D-ribofuranosyl)-1,2,4-triazole-3-carboxamide (142 mg, 0.5 mmol) and difluoromethyl phosphonate (70 mg. 0.6 mmol) were co-evaporated with anhydrous pyridine (3×5 mL) under reduced pressure and taken in 3 mL of anhydrous pyridine. To the above solution under argon atmosphere was added DCC (210 mg, 1.0 mmol). The mixture was stirred at 38° C. for 24 hours. Water (5 mL) was added to the mixture after cooling it to room temperature. The dicyclohexylurea precipitated was filtered off. The filtrate was concentrated under reduced pressure and again filtered. After evaporation of the remaining solvent, the concentrate was co-evaporated with toluene to remove traces of pyridine.

The crude product (158 mg) was dissolved in 5 mL of methanol and Dowex 50W×8-100 resin (1 g) was added. The mixture was heated at 50° C. for 2 hours, filtered, washed with water repeatedly. The filtrate was concentrated to get a viscous residue that was purified on C₁₈ column. The fractions collected were lyophilized to give 100 mg of the titled compound (7).

### Example 6

### 1-(5-O-Vinylphosphonyl-β-D-ribofuranosyl)-1,2,4-triazole-3-carboxamide (8)

1-(2,3-O-Isopropylidene-β-D-ribofuranosyl)-1,2,4-triazole-3-carboxamide (142 mg, 0.5 mmol) and vinylphosphonic acid (58 mg, 0.6 mmol) were co-evaporated with anhydrous pyridine (3×5 mL) under reduced pressure and taken in 3 mL of anhydrous pyridine. To the above solution under argon atmosphere was added DCC (202 mg, 1.0 mmol). The mixture was stirred at 38° C. for 24 hours. A similar work-up procedure as described for Example ˜3 yielded a crude nucleotide derivative.

The crude product was subjected to a similar deprotection and HPLC purification as described for Example 3. The fractions collected were lyophilized to yield 55 mg of the titled compound (8).

### Example 7

### 1-(5-O-(Phenylphosphonyl-β-D-ribofuranosyl)-1,2,4-triazole-3-carboxamide (9)

1-(2,3-O-Isopropylidene-β-D-ribofuranosyl)-1,2,4-triazole-3-carboxamide (142 mg, 0.5 mmol) and phenylphosphonic acid (80 mg, 0.6 mmol) were co-evaporated with anhydrous pyridine (3×5 mL) under reduced pressure and taken in 3 mL of anhydrous pyridine. To the above solution under argon atmosphere was added DCC (202 mg, 1.0 mmoll). The mixture was stirred at 38° C. for 24 hours. A similar work-up procedure as described for Example 3 gave a crude nucleotide derivative.

The crude product was subject to similar deprotection and HPLC purification to give 109 mg of the titled compound (9).

### Example 8

### 5-Ethynyl-1-(5-methylphopsphonyl-β-D-ribofuranosyl)imidazole-4-carboxamide (10)

Step A: 5-Ethynyl-1-(2,3-O-isopropylidene-β-D-ribofuranosyl)imidazole-4-carboxamide

To a stirred suspension of 5-ethynyl-1-β-D-ribofuranosylimidazole-4-carboxamide (200 mg, 0.75 mmol) in 80 mL of dry acetone at 0° C. under argon was added drop-wise 0.02 mL of 70% perchloric acid. The mixture was warmed to room temperature and stirred for 50 minutes. Perchloric acid in the above mixture was carefully neutralized using an equimolar amount of ammonia solution in an ice bath. Solvent was evaporated and the residue was purified on a silica gel column with 10% methanol in chloroform to give 160 mg of the product.

Step B: 5-Ethynyl-1-(5-O-methylphosphonyl-β-D-ribofuranosyl)imidazole-4-carboxamide (10)

The product from Step A (100 mg, 0.33 mmol) was co-evaporated with anhydrous pyridine (3×5 mL) under reduced pressure and taken in 5 mL of anhydrous pyridine. To the above solution under argon atmosphere was added 161 mg (0.78 mmol) of dicyclohexylcarbodiimide (DCC) followed by 38 mg (0.39 mmol) of methylphosphonic acid. The mixture was stirred at 38° C. for 36 hours. A similar work-up procedure as described for Example 3 gave a crude nucleotide derivative.

The crude product was subject to similar deprotection and HPLC purification to give 14 mg of the titled compound (10).

### Example 9

### 5-Ethynyl-1-[5-O-(fluoromethyl)phosphonyl-β-D-ribofuranosyl]imidazole-4-carboxamide (11)

5-Ethynyl-1-(2,3-O-isopropylidene-β-D-ribofuranosyl)imidazole-4-carboxamide (60 mg, 0.195 mmol) and fluoromethylphosphonic acid (26 mg, 0.205 mmol) were co-evaporated with anhydrous pyridine (3×5 mL) under reduced pressure and taken in 3 mL of anhydrous pyridine. To the above solution under argon atmosphere was added 97 mg (0.47 mmol) of DCC. The mixture was stirred at 38° C. for 24 hours. A similar work-up procedure as described for Example 3 gave a crude nucleotide derivative.

The crude product was subject to similar deprotection and HPLC purification to give 9.3 mg of the titled compound (11).

### Example 10

### 1-[5-O-(Difluoromethyl)phosphonyl-β-D-ribofuranosyl]-5-ethynylimidazole-4-carboxamide (12)

Step A: Diflouromethylphosphonic Acid

Diethyl difluoromethylphosphonate (500 mg, 2.66 mmol) and 0.88 mL 96.66 mmol) bromotrimethylsilane were refluxed in 10 mL of anhydrous methylenechloride for 15 hours. The solvent was evaporated and the residue repeatedly co-evaporated with methanol to remove the volatiles. The residue obtained (300 mg) was dissolved in 2 mL of anhydrous pyridine to make a stock solution and stored under argon at −20° C.

Step B: 1-(5-O-Difluoromethylphosphonyl-β-D-ribofuranosyl)-5-ethynylimidazole-4-carboxamide (12)

5-Ethynyl-1-(2,3-O-isopropylidene-1-β-D-ribofuranosyl)imidazole-4-carboxamide (60 mg, 0.195 mmol) and 27 mg (0.205 mmol of difluoromethyl phosphonate were co-evaporated with anhydrous pyridine (3×5 mL) under reduced pressure and taken in 3 mL of anhydrous pyridine. To the above solution under argon atmosphere was added 97 mg (0.47 mmol) of DCC. The mixture was stirred at 38° C. for 24 hours. A similar work-up procedure as described for Example 3 gave a crude nucleotide derivative.

The crude product was subject to similar deprotection and HPLC purification to give 14.1 mg of the titled compound (12).

### Example 11

### 5-Ethynyl-1-(5-O-phosphonomethyl-β-D-ribofuranosyl)imidazole-4-carboxamide (15)

To a stirred solution of 5-ethynyl-1-(2,3-O-isopropylidene-β-D-ribofuranosyl)imidazole-4-carboxamide (125 mg, 0.406 mmol) in 15 mL of anhydrous THF at −78° C. under argon was added slowly a solution of (diethoxyphospinyl)methyl triflate (180 mg, 0.60 mmol, prepared according to a published procedure (Xu et al., J. Org. Chem. 1996, 61, 7697-7701) in 3 mL of anhydrous THF. The mixture was stirred at −78° C. for 1 hour and then evaporated under reduced pressure. The residue was taken in 20 mL of chloroform and washed with 10 mL of water. The chloroform layer was dried over anhydrous magnesium sulfate, filtered and concentrated to dryness to give 110 mg of a crude nucleotide derivative.

To a solution of 110 mg (0.22 mmol) of the crude in 3 mL of anhydrous acetonitrile and dimethylformamide (1:1) under argon was added 0.12 mL (0.87 mmol) of bromotrimethylsilane. The mixture was stirred at room temperature for 12 hours. Solvent was evaporated and the residue was co-evaporated with 5 mL of methanol twice. The residue was taken in 3 mL of water and stirred for 2 hours. The mixture was subjected to purification using C₁₈ column on HPLC. Lyophilization of collected fractions afforded 11.9 mg of the titled compound (15).

### Example 12

### 1-[5-O-(Dihydroxyphosphinyl)methyl-β-D-ribofuranosyl]-1,2,4-triazole-3-carboxamide (19)

Step A: Methyl 1-β-D-ribofuranosyl-1,2,4-triazol-3-carboxylate

Methyl 1-(2,3,5-tri-O-acetyl-β-D-ribofuranosyl)-1,2,4-triazole-3-carboxylate (3.8 g, 10 mmol) was dissolved in anhydrous methanol (50 mL) and sodium methoxide (25 wt. % in methanol, 12 mL) was added. The mixture was stirred at room temperature for 6 h and neutralized with DOWEX 50WX8-100 ion-exchange resin. The resin was filtered through a short pad of cotton, washed with methanol repeatedly. Methanol solution was evaporated to 2.5 g of a crude, titled compound.

Step B: Methyl 1-(2,3-isopropylidene-β-D-ribofuranosyl)-1,2,4-triazole-3-carboxylate (17)

To a suspension of methyl 1-β-D-ribofuranosyl-1,2,4-triazole-3-carboxylate (1.3 g 0.5 mmol) in dry acetone (20 mL) and dimethoxypropane (18 mL) at 0° C. under argon was added drop-wise 0.2 mL of 70% perchloric acid. The mixture was warmed to room temperature and stirred for 50 minutes. Perchloric acid in the above mixture was carefully neutralized using an equimolar amount of ammonia solution in an ice bath. Solvent was evaporated and the residue was loaded on a silica gel column and eluted with 10% methanol in chloroform to give 980 mg of the titled compound.

Step C: Methyl 1-[5-O-(diethoxyphoshinyl)methyl-2,3-isopropylidene-β-D-ribofuranosyl)-1,2,4-triazole-3-carboxylate (18)

A solution of methyl 1-(2,3-O-isopropylidene-β-D-ribofuranosyl)-1,2,4-triazole-3-carboxylate (600 mg, 2 mmol) and sodium hydride (100 mg) in anhydrous THF at −78° C. under argon was stirred for 30 minutes, followed by a slow addition of a solution of (diethoxyphosphinyl)methyl triflate (300 mg, 1 mmol) in THF (10 mL). The mixture was brought to room temperature for 1 h and neutralized with acetic acid, then evaporated under reduced pressure. The residue was taken in 20 mL of chloroform and washed with 10 mL water. The chloroform layer was dried over anhydrous magnesium sulfate, filtered, and concentrated to dryness. The residue was purified on silica gel column chromatography to give 400 mg of the titled compound (18).

Step D: 1-[5-O-(dihydroxyphosphinyl)methyl-β-D-ribofuranosyl]-1,2,4-triazole-3-carboxamide (19)

A solution of compound (18) (400 mg) in 25 mL of methanolic ammonia in a steel vessel stood at room temperature overnight. Ammonia and methanol were evaporated and the residue was purified on silica gel column with 13% methanol in dichloromethane to give 380 mg of 1-[5—(diethoxyphosphinyl)methyl-2,3-O-isopropylidene-β-D-ribofuranosyl]-1,2,4-triazole-3-carboxamide.

To a stirred solution of 1-[5-O-(diethoxyphosphinyl)methyl-2,3-O-isopropylidene-β-D-ribofuranosyl]-1,2,4-triazole-3-carboxamide (350 mg) in acetonitrile (25 mL). was added bromotrimethylsilane (3 mL) and the resulting mixture was stirred at room temperature for 15 h and evaporated to a yellowish syrup, which was dissolved in 5 mL of methanol and concentrated to dryness. This evaporation was repeated three times. The residue was redissolved in 20 mL of methanol and DOWEX 50WX8-100 ion-exchange resin (1 g) was added and the mixture was heated at 50° C. for 2 hours, filtered, washed with water thoroughly. The filtrate was concentrated to get a viscous residue which was purified on C₁₈ column to give 50 mg of the titled compound (19).

### Example 13

### 1-(5-Deoxy-5-S-methylphosphonyl-5-thio-β-D-ribofuranosyl)-1,2,4-triazole-3-carboxamide (23)

Step A: 1-(5-Acetylthio-5-deoxy-2,3-O-isopropylidene-β-D-ribofuranosyl)-1,2,4-triazole-3-carboxamide (20)

Diisopropylazodicarboxylate (1.53 mL, 7.74 mmol) and triphenylphosphine (2.03 g, 7.74 mmol) were dissolved in anhydrous THF (20 mL) at 0° C. After a white precipitate appeared, and 1 g (3.52 mmol) of 2′,3′-O-isopropylidene ribavirin in 15 mL of anhydrous THF and 0.56 mL of thiolacetic acid in 5 mL of anhydrous THF were added simultaneously. The mixture was allowed to warm to room temperature and stirred for 5 hours. Triethylamine was used to neutralize excess thiolacetic acid. Solvent was removed under reduced pressure and the residue was taken in 30 mL of ethyl acetate and washed with 30 mL of water. The aqueous layer was extracted with ethyl acetate (2×20 mL). The combined organic layer was washed with 30 mL of brine and dried over anhydrous magnesium sulfate, filtered and evaporated. The residue was loaded on a silica gel column and the faster moving impurities were eluted using 10:1 and 5:1 chloroform:THF, respectively. The product was eluted using 10:1 chloroform:methanol. Evaporation of the solvent afforded 600 mg of the 5′-acetylthio nucleoside (20).

Step B: 1-(5-Deoxy-2,3-O-isopropylidene-5-thio-β-D-ribofuranosyl)-1,2,4-triazole-3-carboxamide (21)

A 9:1 (v/v) mixture of methanol and triethylamine (7.5: mL) was bubbled with argon at room temperature for 15 minutes and then 200 mg (0.56 mmol) of compound (20) and 2 equivalents of dithiothreitol were added. The mixture was stirred at room temperature for 5 hours. Solvent was evaporated under argon atmosphere and the residue was loaded on a silica gel column. The impurities were eluted using 50:1 methylenechloride:methanol and then the product using 30:1 methylenechloride:methanol. Evaporation of the solvent afforded 130 mg of the 5′-thio nucleoside (21).

Step C: 1-(5-Deoxy-2,3-O-isopropylidene-5-methylphosphonyl-5-thio-β-D-ribofuranosyl)-1,2,4-triazole-3-carboxamide (22)

Compound (21) (100 mg, 0.33 mmol) was co-evaporated with anhydrous pyridine (3×5 mL) under reduced pressure and taken in 5 mL of anhydrous pyridine. To the above solution under argon atmosphere was added 137 mg (0.66 mmol) of dicyclohexylcarbodiimide, followed by 36 mg (0.37 mmol) of methylphosphonic acid. The mixture was stirred at 35° C. for 24 hours. Water (5 mL) was added to the mixture after cooling it to room temperature. The resulting dicyclohexylurea was filtered off. The filtrate was concentrated under reduced pressure and again filtered. After evaporation of the remaining solvent, the concentrate was co-evaporated with toluene to remove traces of pyridine.

Step D: 1-(5-Deoxy-5-methylphosphonyl-5-thio-β-D-ribofuranosyl)-1,2,4-triazole-3-carboxamide (23)

The crude product (22) from Step C was dissolved in 5 mL of methanol and DOWEX 50WX8-100 ion-exchange resin (1 g) was added. The mixture was heated at 50° C. for 2 hours, filtered, washed with water thoroughly. The filtrate was concentrated and purified on reverse-phase HPLC to give 6.2 mg of the titled compound (23).

### Example 14

### 1-(5-Deoxy-5-S-phosphonomethyl-5-thio-1-β-D-ribofuranosyl)-1,2,4-triazole-3-carboxamide (24)

To a solution of 1-(5-deoxy-2,3-O-isopropylidene-5-thio-β-D-ribofuranosyl)-1,2,4-triazole-3-carboxamide (21) (150 mg, 0.50 mmol) in 5 mL of anhydrous DMF at −20° C. was added 20 mg (0.50 mmol) of 60% sodium hydride, followed by addition of 223 mg (0.74 mmol) of (di-O-ethyl)phosphonomethyl trifluoromethanesulfonate. The mixture was stirred at this temperature for 1.5 hours and then solvent was evaporated. The residue was dissolved in-25 mL of ethyl acetate and then washed with water and brine. The organic phase was separated, dried over MgSO4, filtered, and evaporated. The resulting residue (156 mg) was dissolved in 15 mL of anhydrous methylene chloride and to this solution was added 1 mL of bromotrimethylsilane and the mixture was stirred under an inert atmosphere at room temperature for 12 hours. After evaporation of the solvent the residue was dissolved in 20 mL of a 1:1 mixture of methanol and water. The mixture was stirred at 50° C. for 3 hours and concentrated. Chromatography on reverse phase HPLC afforded 13.5 mg of the titled compound (24).

### Example 15

### 1-(5-Deoxy-5-C-sulfo-β-D-ribofuranosyl)-1,2,4-triazole-3-carboxamide (27)

Step A: 1-(5-Deoxy-5-iodo-β-D-ribofuranosyl)-1,2,4-triazole-3-carboxamide (26)

To a solution of 1.7 g (6.5 mmol) of triphenylphosphine in 10 mL of pyridine was added 1.52 g (6.0 mmol) of iodine and the mixture was stirred at room temperature for 20 minutes. To this mixture was added 1-β-D-ribofuranosyl-1,2,4-triazole-3-carboxamide (976 mg, 4.0 mmol). The mixture was stirred at room temperature for 2 hours. Pyridine was evaporated under reduced pressure and co-evaporated with 15 mL of toluene twice. Chromatography on silica gel column with methylenechloride/methanol (20:1 to 7.5:1) yielded 1.1 g of the titled compound (26).

Step B: 1-(5-Deoxy-5-C-sulfo-β-D-ribofuranosyl)-1,2,4-triazole-3-carboxamide (27)

To a solution of compound 26 (600 mg, 1.69 mmol) in 25 mL of 20% methanol in water was added 282 mg (2.28 mmol) of sodium sulfite. The mixture was refluxed for 24 hours. After cooling to room temperature the mixture was filtered and concentrated to 5 ml. The product was purified on reverse-phase HPLC and lyophilized to give 282 mg of the titled compound (27).

### Example 16

### 1-(5-Deoxy-5-thio-β-D-ribofuranosyl)-1,2,4-triazole-3-carboxamide 5′-phosphothioate (28)

A solution of compound 26 (89 mg, 0.25 mmol) and sodium dithiophosphate (400 mg, 2.03 mmol) in 5 mL of water was stirred at room temperature for 36 hours.

Chromatography on reverse-phase HPLC and subsequent lyophilized yielded 2.1 mg of the title compound (28).

### Example 17

### 1-(5-deoxy-5-N-phosphonomethylamino-1-β-D-ribofuranosyl)-1,2,4-triazole-3-carboxamide (32)

Step A: 1-(5-Azido-5-deoxy-1-β-D-ribofuranosyl)-1,2,4-triazole-3-carboxamide (29)

To a solution of 1.5 g (3.65 mmol) of 1-(5-deoxy-5-iodo-β-D-ribofuranosyl)-1,2,4-triazole-3-carboxamide (26) in 15 mL of dimethylformamide was added 3.65 g (5.35 mmol) of sodium azide and the mixture was heated at 90° C. for 12 hours. After evaporation of the solvent the residue was adsorbed on silica gel and loaded on a silica gel column. The product was eluted using 10:1 methylenechloride:methanol. Evaporation of the solvent afforded 1.2 g of the azido compound (29).

Step B: 1-(5-Amino-5-deoxy-1-β-D-ribofuranosyl)-1,2,4-triazole-3-carboxamide (30)

To a solution of compound (29) (1 g, 3.77 mmol) in 50 mL of methanol was added 200 mg of 10% Pd on charcoal. The mixture was shaken at 30 psi hydrogen for 18 hours. The catalyst was filtered and evaporated under reduced pressure to give a crude residue (30) (600 mg).

Step C: 1-15-deoxy-5-N-(di-O-ethyl)phosphonomethylamino-1-β-D-ribofuranosyl]-1,2,4-triazole-3-carboxamide (31)

A solution of the crude (30) (150 mg, 0.62 mmol) in a mixture of 5 mL of anhydrous DMF and 5 mL of anhydrous pyridine at 0° C. and was added 279 mg 90.93 mmol) of (di-O-ethyl)phosphonomethyl trifluoromethanesulfonate). The mixture was stirred at this temperature for 1.5 hours and then solvent was evaporated. The residue was dissolved in 25 mL ethyl acetate and then washed with 15 mL of water and 15 mL brine. The organic phase was separated, dried over MgSO4, filtered and evaporated to give 142 mg of a crude (31).

Step D: 1-(5-Deoxy-5-N-phosphonomethylamino-1-β-D-ribofuranosyl)-1,2,4-triazole-3-carboxamide (32)

To a solution of the crude product (31) was dissolved in 15 mL of anhydrous methylene chloride was added 1 mL (7.5 mmol) of bromotrimethylsilane and the mixture was stirred under an inert atmosphere at 40° C. 15 hours. After evaporation of the solvent the residue was dissolved in 5 mL of water and purified on reverse-phase HPLC. Lyophilization yielded 13.5 mg of the titled compound (32).

### Example 18

### 1-(5-O-Fluorophosphonyl-1-β-D-ribofuranosyl)-1,2,4-triazole-3-carboxamide (35)

Step A: 1-(5-O-tributyldimethylsilyl-2,3-di-O-benzoyl-1-β-D-ribofuranosyl)-1,2,4-triazole-3-carboxamide

A solution of 1-β-D-ribofuranosyl-1,2,4-triazole-3-carboxamide (2.4 g, 10.0 mmol) and tert-butyldimethylchlorosilane (1.65 g 11.0 mmol) in anhydrous pyridine (30 mL) were stirred at room temperature overnight. After the completion of the reaction, the mixture was poured into saturated sodium bicarbonate solution, extracted with ethyl acetate, dried over sodium sulfate, and evaporated. The crude material was redissolved in pyridine (25 mL). Benzoyl chloride (2.6 mL, 22.0 mmol) was added and the resulting mixture was stirred for 30 min. Saturated bicarbonate solution (100 mL) was added and the mixture was extracted with ethyl acetate. The ethyl acetate layer was dried over anhydrous sodium sulfate and evaporated to dryness. The residue was purified on silica gel column chromatography using 2% methanol in dichloromethane to the titled compound (5.2 g).

Step B: 1-(2,3-Di-O-benzoyl-1-β-D-ribofuranosyl)-1,2,4-triazole-3-carboxamide (33)

The product from Step A (2.8 g, 5.0 mmol) was dissolved in 25 mL of tetrahydrofuran. TBAF 1 M solution in THF (15 mL) was added. Reaction mixture was stirred at room temperature overnight. Saturated bicarbonate solution (100 mL) was added and the mixture was extracted with ethyl acetate. The ethyl acetate layer was dried over anhydrous sodium sulfate and evaporated to dryness. The residue was purified on silica gel column chromatography using 15% methanol in dichloromethane to give the titled compound (33) (1.5 g).

Step C: 1-(5-O-Fluorophosphonyl-1-β-D-ribofuranosyl)-1,2,4-triazole-3-carboxamide (35)

Compound (33) (226 mg, 0.5 mmol) and fluorophosphonic acid (55 mg, 0.6 mmol) were co-evaporated with anhydrous pyridine (3×5 mL) under reduced pressure and taken in 5 mL of anhydrous pyridine. To the above solution under argon atmosphere was added DCC (202 mg). The mixture was stirred at 38° C. for 24 hours. Water (3 mL) was added to the mixture after cooling it to room temperature. The resulting dicyclohexylurea precipitate was filtered off. The filtrate was concentrated under reduced pressure and again filtered. After evaporation of the remaining solvent, the concentrate was co-evaporated with toluene to remove traces of pyridine. The residue was treated with 28% aqueous ammonia (3 mL) and stirred for 2 h and evaporated. The crude mixture was purified on reverse-phase HPLC to give 125 mg of the titled compound (35).

### Example 19

### 1-(5-O-hydrogenphosphonyl-β-D-ribofuranosyl-1,2,4-triazole-3-carboxamide (36)

Compound (33), (226 mg, 0.5 mmol) was co-evaporated with anhydrous pyridine (3×5 mL) under reduced pressure and taken in 3 mL of anhydrous pyridine. Diphenyl hydrogen phosphonate (349 mg, 1.5 mmol) was added to the reaction mixture and stirred at room temperature for 15 min. The reaction mixture was quenched by addition of water-triethylamine (1:1, v/v, 5 mL) and stirred for 1 min. The reaction mixture was concentrated under reduced pressure and the residue treated with 50% aqueous methylamine (10 mL) for 1 h. After evaporation of the solvents under vacuum, the oily residue was purified on reverse-phase HPLC to give 75 mg of the titled compound (36).

### Example 20

### 1-[5-Deoxy-5-(dihydroxyphosphinyl)-β-D-ribofuranosyl]-1,2,4-triazole-3-carboxamide (39)

Step A: 1-(2,3-Di-O-benzoyl-5-deoxy-5-iodo-β-D-ribofuranosyl)-1,2,4-triazole-3-carboxamide (37)

A solution of 1-(5-deoxy-5-iodo-β-D-ribofuranosyl)-1,2,4-triazole-3-carboxamide (1.8 g, 5.0 mmol) was dissolved in anhydrous pyridine (10 mL) was cooled to 0° C. and benzoyl chloride (1.3 mL, 11.0 mmol) was added. After 1 h at same temperature, the mixture was poured into saturated sodium bicarbonate and extracted with ethyl acetate. The ethyl acetate phase was dried over sodium sulfate and evaporated. The crude was purified on silica gel column chromatography using 3% methanol in dichloromethane to give 2.1 g of the titled compound (37).

Step B: 1-[5-Deoxy-5-(diethoxyphosphinyl)-2,3-di-O-benzoyl-β-D-ribofuranosyl]-1,2,4-triazole-3-carboxamide (38)

Compound (37) (1.6 g) was dissolved in trimethyl phosphite (5 mL) and heated to 100° C. for 50 h. Excess reagent was evaporated to dryness under high vacuum and the residue was adsorbed on small amount of silica gel. Adsorbed silica gel was loaded on silica gel column and eluted with 3% methanol in dichloromethane to give 500 mg of the titled compound (38).

Step C: 1-[5-Deoxy-5-(dihydroxyphosphinyl)-β-D-ribofuranosyl]-1,2,4-triazole-3-carboxamide (39)

Compound (38) (500 mg, 0.9 mmol) was dissolved in dimethylformamide and acetonitrile (1:1, 10 mL) and bromotrimethylsilane (0.60 mL, 4.5 mmol) was added. The reaction mixture was stirred for 6 h at room temperature and then concentrated under high vacuum. The residue was co-evaporated with methanol and toluene three times. Aqueous ammonia (28%, 15 mL) was added to the residue and stirred at room temperature for 6 h. After evaporation of aqueous solution, the crude residue was purified on a reverse-phase HPLC. The fractions collected were lyophilized to give 50 mg of title compound (39).

### Example 21

### 1-[5-Deoxy-5-(hydroxyl-H-phosphinyl)-β-D-ribofuranosyl]-11,24-triazole-3-carboxamide (40)

A mixture of ammonium phosphinate (0.4 g. 5.0 mmol) and 1,1,1,3,3,3 hexamethyldisilazane (1.07 mL, 5.0 mmol) was heated at 100° C. for 2 h under argon atmosphere. The resulting reagent bis(trimethylsilyl)phosphonite was cooled to 0° C. and 1-(2,3-di-O-benzoyl-5-iodo-β-D-ribofuranosyl)-1,2,4-triazole-3-carboxamide (37) (560 mg, 1.0 mmol) in 20 mL of dichloromethane was added. The reaction mixture was stirred at room temperature over night, filtered and concentrated. The oily residue was dissolved in 5 mL of dichloromethane and 5 mL of methanol, stirred for 2 h at room temperature and evaporated. Aqueous ammonium hydroxide solution (28%, 10 mL) was added to the oily residue and stirred at room temperature for 4 h. The mixture was concentrated to dryness and purified on reverse-phase HPLC. The fractions collected was lyophilized to get 25 mg of the titled compound (40).

### Example 22

### 4-Carbamoyl-1-[5-deoxy-5-(dihydroxyphosphinyl)-β-D-ribofuranosyl]-1,3-imidazolium-5-olate (44)

Step A: 4-Carbamoyl-1-[5-deoxy-5-(dihydroxyphosphinyl)-2,3-O-dibenzoyl-β-D-ribofuranosyl]-1,3-imidazolium-5-olate (43)

A suspension of 4-carbamoyl-1,3-imidazolium-5-olate (188 mg, 1.48 mmol) and sodium sulfate (20 mg) in hexamethyldisilazane (3 mL) and anhydrous xylene (3 mL) was heated under reflux for 3 h and converted to a clear solution. After evaporation of the volatiles, the residue was dried under high vacuum for 30 min, then dissolved in 4 mL of anhydrous dichloroethane. Stannic tetrachloride (140 μL, 1.18 mmol) was added, and followed by addition of compound (41) (700 mg, 1.33 mmol) in dichloroethane (2 mL) and trimethylsilyl triflate (85 μL, 0.44 mmol). The resulting mixture was stirred at room temperature under argon for days, cooled with ice, diluted with chloroform (43).

Step B: 4-Carbamoyl-1-[5-deoxy-5-(dihydroxyphosphinyl)-β-D-ribofuranosyl]-1,3-imidazolium-5-olate (44)

A solution of compound (43) (203 mg, 0.337 mmol) and bromotrimethylsilane (144 μL, 1.1 mmol) in anhydrous acetonitrile (1 mL) stood at room temperature for 12 hours and concentrated to dryness. The residue was dissolved in saturated methanolic ammonia and stirred at room temperature for 12 hours. After evaporation of volatiles the residue was subject to purification on reverse-phase HPLC to yield 21.2 mg of the titled compound (44).

### Example 23

### 1-15,6-Dideoxy-6,6-difluoro-6-(dihydroxyphosphinyl)-β-D-allofuranosyl]-1,2,4-triazole-3-carboxamide (48)

Step A: Methyl 1-[2-O-acetyl-3-O-benzyl-5,6-dideoxy-6-(diethoxyphosphinyl)-6,6-difluoro-β-D-allofuranosyl]-1,2,4-triazole-3-carboxylate

Methyl-1,2,4-triazole-4-carboxylate (300 mg, 2.5 mmol) in 1,1,1,3,3,3 hexamethyldisilazane (HMDS, 5 mL) was refluxed in presence of catalytic amount of ammonium sulfate (5 mg). Excess HMDS was evaporated under high vacuum. The resulting silylated triazole base was redissolved in anhydrous acetonitrile and 1, 2-di-O-acetyl-3-O-benzyl-5,6-dideoxy-6-(diethoxyphosphinyl)-6,6-difluoro-β-D-allofuranose (1.25 g, 2.5 mmol), synthesized according to a reported procedure (Matulic-Adamic et al., J. Org. Chem; 1995, 60, 2563-2569), was added. After addition of Tin (IV) chloride (0.9 mL, 7.5 mmol) the reaction mixture was heated under reflux for 2 h. After cooling to room temperature, the mixture was diluted with chloroform, filtered through celite, and washed with saturated sodium bicarbonate solution. The organic layer was dried over anhydrous sodium sulfate and evaporated. The crude product was purified on silica gel column chromatography using 5% methanol in dichloromethane to give 1.1 g of the titled compound (46) along with its regioisomer: methyl 1-[2-O-acetyl-3-O-benzyl-5,6-Dideoxy-6-(diethoxyphosphinyl)-6,6-difluoro-β-D-allofuranosyl]-1,2,4-triazole-5-carboxylate (50 mg).

Step B: 1-[3-O-benzyl-5,6-dideoxy-6-(diethoxyphosphinyl)-6,6-difluoro-β-D-allofuranosyl]-1,2,4-triazole-3-carboxamide (47)

A solution of compound (46) (1.0 g) and methanolic ammonia saturated at 0° C. in a steel vessel stood at room temperature overnight. Excess ammonia was allowed to evaporate. After evaporation of methanol under reduced pressure, a solid crude product 1-[3-O-benzyl-5,6-dideoxy-6-(diethoxyphosphinyl)-6,6-difluoro-β-D-allofuranosyl]-1,2,4-triazole-3-carboxamide (47) (725 mg) was obtained.

Step C: 1-[5,6-Dideoxy-6-(dihydroxyphosphinyl)-6,6-difluoro-β-D-allofuranosyl]-1,2,4-triazole-3-carboxamide (48)

Compound (47) (505 mg, 1.0 mmol) from the step B was dissolved in anhydrous dichloromethane (25 mL) and the mixture was cooled to −78° C. Boron trichloride (2 M in dichloromethane, 2.1 mL) was added. The reaction mixture was brought to room temperature and stirred for 1 h. Methanol (10 mL) was added and evaporated. This process was repeated three times and the residue was taken in acetonitrile and DMF (1:1 v/v, 20 mL). Then, to the mixture was added bromotrimethylsilane (2.2 mL, 16.8 mmol) under argon atmosphere and stirred at room temperature. After 40 h, the mixture was evaporated to a reddish, oily residue and co-evaporated with methanol (3×10 mL) and toluene (3×10 mL). The crude product was purified on a reverse-phase (C18) HPLC to give 50 mg of the titled compound (48).

### Example 24

### 4-Carbamoyl-1-[5,6-dideoxy-6-(dihydroxyphosphinyl)-6,6-difluoro-β-D-allofuranosyl]-1,3-imidazolium-5-olate (50)

Step A: Carbamoyl-1-[2-O-acetyl-3-O-benzyl-5,6-dideoxy-6-(diethoxyphosphinyl)-6,6-difluoro-β-D-allofuranosyl]-1,3-imidazolium-5-olate (49)

4-Carbamoylimidazolium-5-olate ((45), 127 mg 1.0 mmol) in 1,1,1,3,3,3 hexamethyldisilane (HMDS, 5 mL) and xylene (5 mL) was refluxed in presence of catalytic amount of ammonium sulfate (2 mg). Excess HMDS was evaporated under high vacuum. The resulting silylated imidazolium base was dissolved in anhydrous nitromethane and 1,2-O-diacetyl-3-O-benzyl-5,6-dideoxy-6-(diethoxyphosphinyl)-6,6-difluoro-β-D-allofuranose (500 mg, 1.0 mmol), synthesized according to a reported procedure (Matulic-Adamic et al., J. Org. Chem; 1995, 60, 2563-2569), was added. After addition of titanium (IV) chloride (0.15 mL, 1.3 mmol) the reaction mixture was stirred at room temperature for 42 h, poured into a suspension of 4 g of sodium carbonate in methanol. The methanol solution was filtered through celite and evaporated. The residue was purified on silica gel column chromatograpy using 20% methanol, 79.5% ethyl acetate and 0.5% triethylamine to give 280 mg of the titled compound (49).

Step B: 4-Carbamoyl 1-[5,6-dideoxy-6-(dihydroxyphosphinyl)-6,6-difluoro-β-D-allofuranosyl]-1,3-imidazolium-5-olate (50)

Compound (49) (280 mg 0.5 mmol) from the step B was dissolved in anhydrous dichloromethane (25 mL) and the mixture was cooled to −78° C. Boron trichloride (2 M in dichloromethane, 1.05 mL, 2.1 mmol) was added. The reaction mixture was brought to room temperature and stirred for 2 h. Methanol (10 mL) was added and evaporated to dryness. This process was repeated three times and the residue was taken in acetonitrile and DMF (1:1 v/v-20 mL). Then, to the reaction mixture was added bromotrimethylsilane (2.2 mL, 16.5 mmol) under argon atmosphere and stirred at room temperature. After 48 h, the mixture was evaporated to a reddish oily residue and co-evaporated with methanol (3×10 mL) and toluene (3×10 mL). The crude product was purified on a reverse-phase HPLC to give 30 mg of the titled compound (50).

### Example 25

### 1-[5-O-(H-Thiophosphonyl)-β-D-ribofuranosyl]-1,2,4-triazole-3-carboxamide (53)

1-(2,3-Di-O-benozyl-1-β-D-ribofuranosyl)-1,2,4-triazole-3-carboxamide (33) (226 mg. 0.5 mmol) and 9-fluorenemethyl (H)-phosphonothioate (400 mg, 1.5 mmol) were dissolved in 10% pyridine in dichloromethane containing (10 mL). Trimethylacetylchloride (0.07 mL, 0.7 mmol) was added and the mixture was stirred at room temperature for 5 min. Then, triethylamine (10 mL) was added and stirred for further 20 min. The solvent was evaporated under vacuum, and the residue was treated with aqueous methylamine (50%, 5 mL) for 1 hour. The solution was concentrated and purified on a reverse-phase HPLC to give 25 mg of the titled compound (53).

### Example 26

### 1-β-D-ribofuranosyl-1,2,4-triazole-3-carboxamide 5′-dithiophosphorothioate (54)

1-(2,3-Di-O-benzoyl-1-β-D-ribofuranosyl)-1,2,4-triazole-3-carboxamide (33) (226 mg, 0.5 mmol) and 9-fluorenemethyl (H)-phosphonothioate (400 mg, 1.5 mmol) were dissolved in 10% pyridine in dichloromethane (10 mL). Trimethylacetylchloride (0.07 mL) was added and the mixture was stirred at room temperature. After 5 min, solvent was evaporated to an oily residue. The residue was redissolved in dichloromethane containing lutidine (10%, 10 mL) and reacted with sulfur powder (50 mg, 1.5 mmol). After 10 min at room temperature, to the reaction mixture was added pyridine-28% aqueous ammonia (1:1, 15 mL). The reaction mixture was further stirred at room temperature for 24 h, evaporated to an oily residue. After repeated purification on a reverse-phase HPLC 1.2 mg of the titled compound (54) was obtained.

### Example 27

### 1-[5-O-(S-Pivaloyl-2-thioethoxy)methylphosphinyl-β-D-ribofuranosyl]-1,2,4-triazole-0.3-carboxamide (56)

To a solution of 1-(2,3-O-isopropylidene-1-β-D-ribofuranosyl)-1,2,4-triazole-3-carboxamide (190 mg, 0.41 mmol) in anhydrous pyridine (10 mL) under argon were added S-pivaloyl-2-thioethanol (200 mg, 3 equiv.) and 1-(mesitylene-2-sulfonyl)-3-nitro-1,2,4-triazole (243 mg, 2 equiv.). After stirring at room temperature for 2 days, the reaction mixture was neutralized with an aqueous solution of 1 M triethylammonium hydrogencarbonate buffer (pH=7.5), and extracted with chloroform. The organic layer was dried over sodium sulfate, filtered, and evaporated to dryness under reduced pressure. Chromatography on silica with 0-10% methanol in dichloromethane gave 78 mg of the nucleoside 5′-O-(S-pivaloyl-2-thioethyl)methylphosphonate 55.

To a solution of the nucleoside 5′-O-(S-pivaloyl-2-thioethyl)methylphosphonate 55 (70 mg, 0.138 mmol) in methanol (5 mL) was added DOWEX 50WX8-100 ion-exchange resin (prewashed with water and methanol, 200 mg). The reaction mixture was stirred at room temperature overnight. The resin was filtered and washed with methanol and water. The combined solution was evaporated to dryness. The crude residue was chromatographed on silica gel with 0-10% methanol in dichloromethane to yield 44 mg of the titled compound 56.

### Example 28

### 3-Cyano-1-[5-O-(pivaloyloxy)methylphosphinyl-β-D-ribofuranosyl]-1,2,4-triazole (59)

Step A. The preparation of 3-cyano-1-[(5-O-methylphosphinyl)-β-D-ribofuranosyl]-1,2,4-triazole (58)

To a solution of 3-cyano-1-[(2,3-O-isopropylidene-5-O-methylphosphino)-β-D-ribofuranosyl]-1,2,4-triazole (57) (obtained as a minor product from the preparation of compound (4) (150 mg, 0.435 mmol) in methanol (5 mL) was added DOWEX 50WX8-100 ion-exchange resin (prewashed with water and methanol) (500 mg). The reaction mixture was stirred at room temperature for 16 h. The resin was filtered and washed with methanol. The solution was concentrated to dryness to give 110 mg of crude product. The crude product was used in the next step without further purification. 45 mg of the crude was purified by reversed-phase HPLC (C18) to give 26.9 mg of pure compound (58).

Step B. 3-Cyano-1-[5-O-(pivaloyloxy)methylphosphino-β-D-ribofuranosyl]-1,2,4-triazole (59)

A solution of compound (58) (38 mg, 0.125 mmol) and tributylstannyl methoxide (40 mg, 0.125 mmol) in methanol (3 mL) was stirred at 25° C. for 30 min. Methanol was removed by evaporation and the residue was coevaporated with acetonitrile (3×3 mL). To the residue in anhydrous acetonitrile (3 mL) were added tetrabutylammonium bromide (40 mg, 0.125 mmol) and iodomethyl pivalate (151 mg, 0.625 mmol, prepared by reacting chloromethyl pivalate with sodium iodide in acetonitrile). The mixture was refluxed for 1 h, then cooled to room temperature, concentrated to a small volume (0.3 mL) under reduced pressure, and then applied onto a silica gel column. The column was eluted with a mixture of methylene chloride and ethyl acetate. The resulting product was further purified by reversed-phase HPLC (C18) to give 20.4 mg of the titled compound (59).

B. Biological Assays

### Example 29

### Assay for Inhibition of IMPDH Activity

The assays employed to measure the inhibition of inosine monophosphate dehydrogenase (IMPDH) activity are described below. The effectiveness of the compounds of the present invention as inhibitors of IMPDH enzymes was determined in the following assays. This assay was used to measure the ability of the nucleotide mimics of the present invention to inhibit the enzymatic reaction catalyzed by IMPDH enzymes. The assay is useful for measuring the activity of IMPDH from several organisms, including human, fungal, and bacterial isoforms. In the enzymatic reaction, the oxidation of inosine 5′-monophosphate (IMP) to xanthosine 5′-monophosphate (XMP) is coupled to the reduction of nicotinamide adenine dinucleotide (NAD). This reaction is monitored at 340 nm using a UV/VIS spectrophotometer or at 474 nm using a fluorometer (excitation wavelength=344 nm). This assay is a modification of a reported method (W. Wang and L. Hedstrom, “A Potent ‘Fat Base’ Nucleotide Inhibitor of IMP Dehydrogenase,” Biochemistry 1998, 37, 11949-52).

Procedure:

Assay Buffer Conditions: (200 uL-total/reaction)

50 mM Tris-HCl, pH 8.0

100 mM KCl

3 mM EDTA

1 mM DTT

50 uM IMP

150 uM NAD

30 nM purified human type II IMPDH, or

7.5 nM purified Candida albicans IMPDH

The compounds were tested at various concentrations up to 500 uM final concentration. The standard IMPDH assay is performed in a 96-well plate (Corning). An appropriate volume of assay buffer, containing the substrates IMP and NAD, was pipetted into the plate wells. Nucleoside derivatives of the present invention were added to the reactions at the desired concentrations. The reactions were initiated by the addition of enzyme. The reactions were allowed to proceed for 5 minutes at 25° C. The production of NADH was monitored at 340 nm on a microplate spectrophotometer (Molecular Devices Corp, Sunnyvale, Calif.). Initial velocity data (mA min⁻¹) was collected and fit to the equations below. Blank reactions were prepared in parallel with the test reactions in which enzyme was omitted from the reactions, substituted by an appropriate volume of enzyme diluent.

The percentage of inhibition was calculated according to the following equation:

% Inhibition=[1−(mA min⁻¹ in test reaction−mA min⁻¹ in blank)/(mA min⁻¹ in control reaction−mA min⁻¹ in blank)]×100.

Inhibition constants (Kᵢ) were determined for representative compounds that exhibited ≧50% inhibition at 500 uM when tested in the IMPDH inhibition assay. Each inhibitor was titrated over an appropriate range of concentrations, and inhibition constants were determined using the following equations where v=initial velocity, Vₘ=maximal velocity, S=substrate, I=inhibitor, Kₘ=Michaelis constant, and Kᵢ=inhibition constant:

Michaelis-Menten Equation:

v=Vₘ[S]/(Kₘ+[S])

Competitive Inhibition Equation:

v=Vₘ[S]/(Kₘ(1+[I]/Kᵢ)+[S])

Inhibition constants (Kᵢ) for irreversible inhibitors of IMPDH were determined using the following three equations where k_{obs}=observed rate constant, t=time, A=absorbance at time t, A₀=initial absorbance at time zero, V₀=initial rate, S=substrate, I=inhibitor, k₂=dissociation rate constant, Kₘ=Michaelis constant, Kᵢ, ₐₚₚ=apparent inhibition constant, and Kᵢ=inhibition constant:

Irreversible Inhibition Equations 1-3:

A−A₀=V₀[1−exp(−k_{obs}t)] (equation 1)

K_{obs}=k₂[I]/(K_{i, app}+[I]) (equation 2)

Kᵢ=K_{i, app}/(1+[S]/Kₘ) (equation 3)

Representative compounds of the present invention tested in the human IMPDH inhibition assay exhibited inhibition constants less than 250 μM.

TABLE 1
Inhibition of IMPDH by Nucleotide Mimics

| | Kᵢ (μM) | Kᵢ (μM) | % Inhibition at 100 μM |
|---|---|---|---|
| Compound # | Human Type II | C. albicans | C. albicans |
| 1 | 0.94 | 1.34 | |
| 39 | 1.82 | 1.48 | |
| 50 | 2.04 | | 71.5 |
| 44 | 7.92 | | 98.1 |
| 48 | 27.1 | 20.4 | |
| 2 | 34.2 | | 82.0 |
| 53 | 34.7 | 10.7 | |
| 19 | 85 | | |

### Example 30

### Antibacterial Assays

To examine the antimicrobial potential of the nucleotide mimics of the present invention an assay was employed that allowed the screening of a large number of compounds simultaneously. The type of bacteria chosen to screen the compounds are organisms associated with human disease and represent major groups of bacteria based on their structure and metabolism.

Lawn Screening Assay:

Bacterial cultures of Escherichia coli, Staphylococcus aureus and Pseudomonas aeruginosa were incubated overnight at 37° C. in a shaker incubator. A lawn of each overnight bacterial culture was made by plating 200 μl of bacteria on agar plates containing either Nutrient Broth (E. coli, S. aureus) or Tryticase Soy Broth (P. aeruginosa). Immediately after plating, sterile blank paper discs were put on top of the lawn and a compound was applied to each blank paper disc. Plates were then incubated overnight and examined for the inhibition of bacterial growth the following day.

Minimal Inhibitory Concentration Determination

Bacterial cells (2×10⁴) growing in exponential phase were plated in 96-well plates and treated with different concentrations (0-200 μg/ml) of the nucleotide mimics of the present invention. The plates were incubated overnight at 37° C. and then examined spectrophotometrically at 600 nm to determine the minimum concentration of each compound that inhibited replication of bacteria as determined by no increase in absorbance at 600 nm.

### Example 31

### Mammalian Cell Growth Inhibition Assay

The assays employed for determining the cytotoxicity of the nucleotide mimics of the present invention to mammalian cells are described below.

Mammalian Cells and Growth Conditions

Human CCRF-CEM and HepG-2 cells were obtained from American Tissue Culture Collection (ATCC) and grown according to ATCC specifications. Briefly, CCRF-CEM, a lymphoblastoid cell line, was grown and maintained as a suspension culture in RPMI 1640 medium containing 2 mm L-glutamine, 10 mM HEPES, 1 mM sodium pyruvate, 4.5 g/L glucose, 1.5 g/L sodium bicarbonate and supplemented with 10% (v/v) dialyzed and heat-inactivated fetal bovine serum. HepG2, a liver tumor cell line, was grown and maintained as a monolayer in Eagle's Minimum Essential Medium with Earle's BSS (MEM/EBSS), 1 mM sodium pyruvate, 0.1 mM non-essential amino acids, 1.5 g/L sodium bicarbonate and supplemented with 10% (v/v) dialyzed and heat-inactivated fetal bovine serum. Both cells lines were grown at 37° C. in a 95% humidified environment and 5% CO₂ atmosphere.

Cytotoxicity Assays: MTT Assay.

The cytotoxicity of the nucleotide mimics of the present invention to mammalian cells was determined by measuring cell survival using 3-(4,5-dimethylthiazol-2-yl)-2,5-diphenyltetrazolium bromide (MTT) (Slater T. F. et al., Biochim. Biophys. Acta 1963, 77, 383; Mossman T. J. Immunol Methods 1983, 65, 55; M. E. et al., 1999, J. Biol. Chem. 28505-13). MTT is a water soluble tetrazolium salt that is converted to an insoluble purple formazan by active mitochondrial dehydrogenases of living cells. Dead cells do not cause this change. Conversion of MTT into the insoluble formazan by non-treated control or treated cells was monitored at 540 nm.

CCRF-CEM and HepG2 cells (3×10⁴) were plated in 96-well plates in either RPMI or MEM/EBSS media, respectively. The next day, cells were incubated with different concentrations (0-200 μM) of the nucleotide mimics of the present invention for 72 hr. Following treatment, MTT (2 mg/ml in PBS) dye was added to each well so that the final concentration was 0.5 mg/ml and then incubated for 4 hr at 37° C. Media and MTT dye were removed without disturbing the cells and 100% DMSO was added to dissolve the precipitate. After a 10 minute incubation at room temperature, the optical density values were measured at 540 nm, using the Spectra Max Plus plate reader. Survival was expressed as the percentage of viable cells in treated samples relative to non-treated control cells.

### Example 32

### Serum Stability Assessment

The stability of nucleotide mimics was assessed in fetal calf serum generally following the procedure outlined in Arzumanov et al., (J. Biol. Chem. 271(40):24389-24394, 1996). Fetal calf serum purchased from HyClone Corporation was mixed 1:1 with each compound containing Tris-HCl buffer and MgCl₂. Typically the total volume used for the experiment was 500 μl.

The final concentrations of the reaction components were as follows:

50 mM Tris-HCl, pH 7.4

0.1 mM MgCl₂

500 μM nucleotide mimic

10% (v/v) fetal calf serum

The reaction mixtures were made up and incubated at 37° C. At appropriate times aliquots of 25 μl were removed and added to 65 μl ice-cold methanol. These solutions were incubated for at least one hour at −20° C. and typically overnight. After incubation samples were centrifuged for at least 20 minutes at high speed in a microcentrifuge. The supernatant was transferred to a clean tube and the extract was dried under vacuum in a LabConco Centrivap Concentrator. The dried extracts were resuspended in dH₂O and filtered to remove particulate before analysis on reverse phase HPLC.

The reverse phase HPLC columns used for the analysis were either a Phenomenex C18 Aqua column (2×100 mm) or the Phenomenex C18 Aqua column (3×150 mm) used with the appropriate guard column. The HPLC was run at 0.2 ml/min (for the 2×100 mm column) or at 0.5 ml/min (for the 3×150 mm column) with the following buffer system: 5 mM tetrabutylammonium acetate, 50 nM ammonium phosphate, and an acetonitrile gradient running from 5% up to as high as 60%. The amount of remaining parent compound at each time point was used to determine the half-life of the compound. Time points were only taken through 24 hours so that if greater than 50% of a compound was still intact after 24 hours incubation the half-life was expressed as >24 hours. Unmodified nucleoside monophosphates were used as positive controls. Under these conditions unmodified nucleoside monophosphates had half-lives of approximately three to six hours.

TABLE 2
Serum Stability of Nucleotide Mimics

| | Serum t_{1/2} |
|---|---|
| Compound Name/Compound No. | (hours) |
| EICAR 5′-monophosphate | 3 |
| Ribavirin 5′-monophosphate | 6 |
| 40 | >24 |
| 48 | >24 |
| 5 | >24 |
| 1 | 18 |
| 10 | >8 |

What is claimed:

1. A compound of Formula (I): 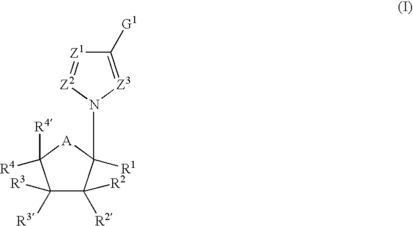
which may be a D- or L-nucleotide; wherein:
A is O, S, CH₂, CHF, CF₂, or NH;
R^{4′} is -L-R⁵ where L is selected from the group consisting of O, S, NH, NR, CH₂, CH₂O, CH₂S, CH₂NH, CH₂NR, CHY, CY₂, CH₂CH₂, CH₂CHY, and CH₂CY₂, where Y is F, Cl, Br, or selected from alkyl, alkenyl, and alkynyl optionally containing one or more heteroatoms;
R⁵ is a moiety of Formula (II) or (III): 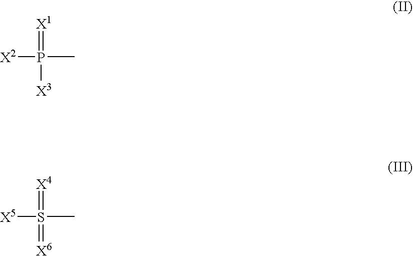
where X¹, X⁴, and X⁶ independently are O, S, NH, or NR; X², X³, and X⁵ are selected independently from the group consisting of H, F, OH, SH, NH₂, NHOH, N₃, CN, ⁻BH₃M⁺, R, OR, SR, NHR, NR₂, and R*, wherein R* is a prodrug substituent;
R¹, R₂, R_{2′}, R³, R^{3′}, and R⁴ are selected independently from a group consisting of H, F, Cl, Br, I, OH, SH, NH₂, NHOH, N₃, NO₂, CHO, COOH, CN, CONH₂, COOR, R, OR, SR, SSR, NHR, and NR₂; alternatively, R² and R^{2′} together and R³ and R^{3′} together independently are ═O, ═S, or =J-Q, where J is N, CH, CF, CCl, or CBr, and Q is H, F, Cl, Br, N₃ or R;
Z¹, Z², and Z³ are independently N, CH or C-G²;
G¹ and G² are selected independently from a group consisting of H, F, Cl, Br, I, OH, SH, NH₂, NHOH, NHNH₂, N₃, NO, NO₂, CHO, COOH, CN, CONH₂, CONHR, C(S)NH₂, C(S)NHR, COOR, R, OR, SR, NHR, and NR₂; when two or more G 2 groups are present on a molecule, they can be same as or different from one another; and
R is selected from the group consisting of alkyl, alkenyl, alkynyl, aryl, acyl, and aralkyl optionally containing one or more heteroatoms;
with provisos that:
(1) at least one of X¹, X², and X³ is not 0, OH or OR, when L is CH₂O which is linked to P through O;
(2) at least one of X¹, X², and X³ is not 0, OH, OC₅H₆ or OCH₂C₅H₆ when L is CH₂CH₂, G¹ is CONH₂, Z¹ and Z³ are N, Z² is CH, R¹, R², R³, R⁴ are H, and R^{2′} and R^{3′} are OH;
(3) one of X² and X³ is not NH₂ when the other of X² and X³ is OH, X¹ is O, L is CH₂O which is linked to P through O, G¹ is CONH₂, Z¹ and Z³ are N, Z² is CH, R¹, R², R³, and R⁴ are H, and R² and R³ are OH;
(4) X⁵ is not NH₂ when X⁴ and X⁶ are O, L is CH₂O which is linked to S through O, G¹ is CONH₂, CSNH₂ or CN, Z¹ and Z³ are N, Z² is CH, R¹, R², R³, and R⁴ are H, and R^{2′} and R^{3′} are OH.
(5) when L is CH₂O linked to P through CH₂ and R⁴ is alkyl, alkoxy, halomethyl, CH₂—O-t-butyldimethylsilyl, CH₂OH, CH₂N₃, CH₂CN, CH₂CH₂N₃, or CH₂CH₂OH, G¹ is not CONHR; and
(6) when L is CH₂CH₂, CH₂O, CH₂S, CH₂CHF, or CH₂CF₂ which is linked to P through CH₂ and R¹, R², R³, and R⁴ are all hydrogen, G¹ is not CONHR.

2. The compound according to claim 1 having Formula (IV): 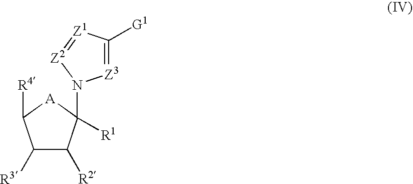
wherein R^{2′} and R^{3′} are independently H, F, or OH.

3. The compound according to claim 1 having Formula (V): 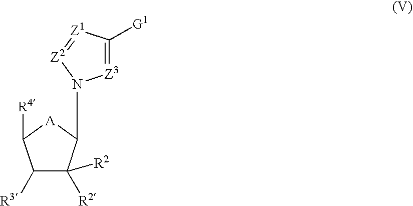
wherein R^{3′} is H, F, or OH.

4. The compound according to claim 1 having Formula (VI): 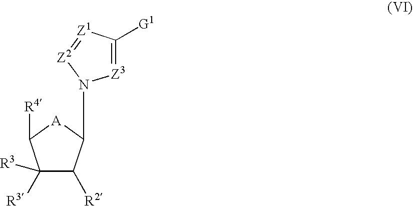
wherein R^{2′} is H, F, or OH.

5. The compound according to claim 1 having Formula (VII): 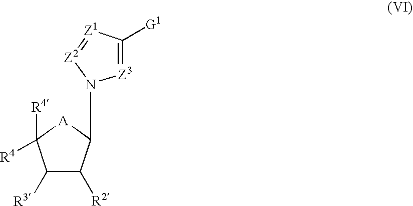
wherein R^{2′} and R^{3′} are independently H, F, or OH.

6. The compound according to claim 1 having Formula (VIII): 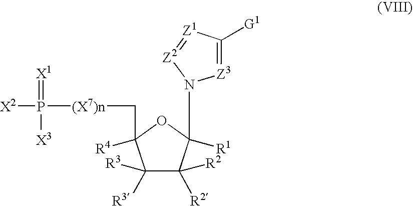
wherein X¹ is O or S;
wherein X² and X³ are selected independently from the group consisting of H, OH, SH, NH₂, F, NHOH, N₃, CN, ⁻BH₃M⁺, NHR, R, OR, SR, and R*;
wherein X⁷ is O, S, NH, NMe, CH₂, CHF, CCl₂, or CF₂; and
wherein n is 0 or 1.

7. The compound according to claim 6 wherein R* is 1,2-O-diacylglyceryloxy, 1,2-O-dialkylglyceryloxy, 1-O-alkyl-2-O-acylglyceryloxy, 1-O-acyl-2-O-alkylglyceryloxy, 1-S-alkyl-2-O-acyl-1-thioglyceryloxy, acyloxymethoxy, S-acyl-2-thioethoxy, S-pivaloyl-2-thioethoxy, acyloxymethoxy, pivaloyloxymethoxy, alkoxycarbonyloxymethoxy, or S-alkyldithio-S′-ethyoxy.

8. The compound according to claim 1 having Formula (IX): 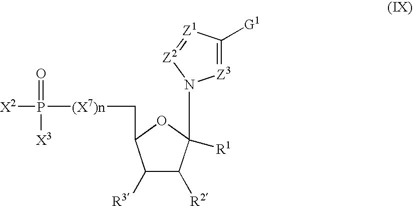
wherein X² and X³ are selected independently from the group consisting of H, F, OH, SH, NH₂, NHOH, N₃, CN, ⁻BH₃M⁺, NHR, R, OR, SR, OR, and R*;
wherein X⁷ is O, S, NH, NMe, CH₂, CHF, CCl₂, or CF₂;
wherein n is 0 or 1; and
wherein R^{2′} and R^{3′} are independently H, F, or OH.

9. The compound according to claim 8 wherein R* is 1,2-O-diacylglyceryloxy, 1,2-O-dialkylglyceryloxy, 1-O-alkyl-2-O-acylglyceryloxy, 1-O-acyl-2-O-alkylglyceryloxy, 1-S-alkyl-2-O-acyl-1-thioglyceryloxy, acyloxymethoxy, S-acyl-2-thioethoxy, S-pivaloyl-2-thioethoxy, acyloxymethoxy, pivaloyloxymethoxy, alkoxycarbonyloxymethoxy, or S-alkyldithio-S′-ethyoxy.

10. The compound according to claim 1 having Formula (X): 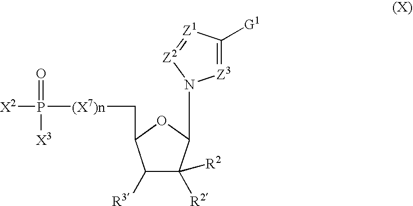
wherein X² and X³ are selected independently from the group consisting of H, F, OH, SH, NH₂, NHOH, N₃, CN, ⁻BH₃M⁺, NHR, R, OR, SR, and R*;
wherein X⁷ is O, S, NH, NMe, CH₂, CHF, CCl₂, or CF₂;
wherein n is 0 or 1; and
wherein R^{3′} is H, F, or OH.

11. The compound according to claim 10 wherein R* is 1,2-O-diacylglyceryloxy, 1,2-O-dialkylglyceryloxy, 1-O-alkyl-2-O-acylglyceryloxy, 1-O-acyl-2-O-alkylglyceryloxy, 1-S-alkyl-2-O-acyl-1-thioglyceryloxy, acyloxymethoxy, S-acyl-2-thioethoxy, S-pivaloyl-2-thioethoxy, acyloxymethoxy, pivaloyloxymethoxy, alkoxycarbonyloxymethoxy, or S-alkyldithio-S′-ethyoxy.

12. The compound according to claim 1 having Formula (XI): 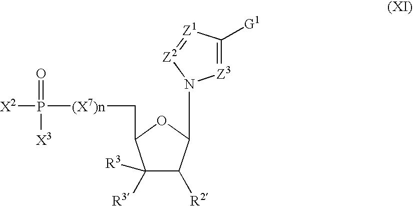
wherein X² and X³ are selected independently from the group consisting of H, F, OH, SH, NH₂, NHOH, N₃, CN, ⁻BH₃M⁺, NHR, R, OR, SR and R*;
wherein X⁷ is O, S, NH, NMe, CH₂, CHF, CCl₂, or CF₂;
wherein n is 0 or 1; and
wherein R^{2′} is H, F, or OH.

13. The compound according to claim 12 wherein R* is 1,2-O-diacylglyceryloxy, 1,2-O-dialkylglyceryloxy, 1-O-alkyl-2-O-acylglyceryloxy, 1-O-acyl-2-O-alkylglyceryloxy, 1-S-alkyl-2-O-acyl-1-thioglyceryloxy, acyloxymethoxy, S-acyl-2-thioethoxy, S-pivaloyl-2-thioethoxy, acyloxymethoxy, pivaloyloxymethoxy, alkoxycarbonyloxymethoxy, or S-alkyldithio-S′-ethyoxy.

14. The compound according to claim 1 having Formula (XII): 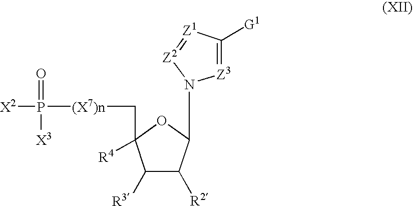
wherein X² and X³ are selected independently from the group consisting of H, OH, SH, F, OH, SH, NH₂, NHOH, N₃, CN, ⁻BH₃M⁺, NHR, R, OR, SR, OR and R*;
wherein X⁷ is O, S, NH, NMe, CH₂, CHF, CCl₂, or CF₂;
wherein n is 0 or 1; and
wherein R^{2′} and R^{3′} are independently H, F, or OH.

15. The compound according to claim 14 wherein R* is 1,2-O-diacylglyceryloxy, 1,2-O-dialkylglyceryloxy, 1-O-alkyl-2-O-acylglyceryloxy, 1-O-acyl-2-O-alkylglyceryloxy, 1-S-alkyl-2-O-acyl-1-thioglyceryloxy, acyloxymethoxy, S-acyl-2-thioethoxy, S-pivaloyl-2-thioethoxy, acyloxymethoxy, pivaloyloxymethoxy, alkoxycarbonyloxymethoxy, or S-alkyldithio-S′-ethyoxy.

16. The compound according to claim 1 having Formula (XIII): 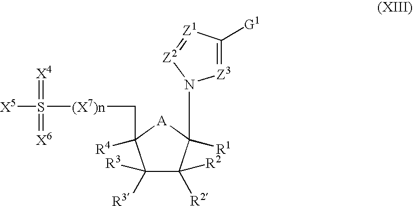
wherein X⁴ and X⁶ are independently O or S;
wherein X⁵ is selected from the group consisting of F, OH, SH, NH₂, NHOH, N₃, CN, ⁻BH₃M⁺, NHR, R, OR, SR, and R*;
wherein X⁷ is O, S, NH, NMe, CH₂, CHF, CCl₂, or CF₂; and
wherein n is 0 or 1.

17. The compound according to claim 16 wherein R* is 1,2-O-diacylglyceryloxy, 1,2-O-dialkylglyceryloxy, 1-O-alkyl-2-O-acylglyceryloxy, 1-O-acyl-2-O-alkylglyceryloxy, 1-S-alkyl-2-O-acyl-1-thioglyceryloxy, acyloxymethoxy, S-acyl-2-thioethoxy, S-pivaloyl-2-thioethoxy, acyloxymethoxy, pivaloyloxymethoxy, alkoxycarbonyloxymethoxy, or S-alkyldithio-S′-ethyoxy.

18. The compound according to claim 1 having Formula (XIV): 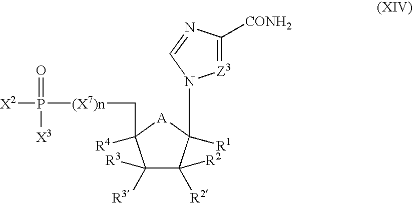
wherein X² and X³ are selected independently from the group consisting of F, OH, SH, NH₂, NHOH, N₃, CN, ⁻BH₃M⁺, NHR, R, OR, SR and R*;
wherein X⁷ is O, S, NH, NMe, CH₂, CHF, CCl₂, or CF₂;
wherein n is 0 or 1; and
wherein Z³ is N, CH, C—OH, or C-ethynyl.

19. The compound according to claim 18 wherein R* is 1,2-O-diacylglyceryloxy, 1,2-O-dialkylglyceryloxy, 1-O-alkyl-2-O-acylglyceryloxy, 1-O-acyl-2-O-alkylglyceryloxy, 1-S-alkyl-2-O-acyl-1-thioglyceryloxy, acyloxymethoxy, S-acyl-2-thioethoxy, S-pivaloyl-2-thioethoxy, acyloxymethoxy, pivaloyloxymethoxy, alkoxycarbonyloxymethoxy, or S-alkyldithio-S′-ethyoxy.

20. The compound according to claim 1 having Formula (XV): 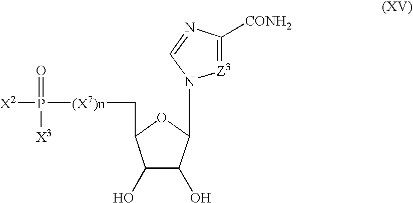
wherein X² and X³ are selected independently from the group consisting of F, OH, SH, NH₂, NHOH, N₃, CN, ⁻BH₃M⁺, NHR, R, OR, SR and R*;
wherein Z³ is N, CH, C—OH, or C-ethynyl;
wherein X⁷ is O, S, NH, NMe, CH₂, CHF, CCl₂, or CF₂; and
wherein n is 0 or 1.

21. The compound according to claim 20 wherein R* is 1,2-O-diacylglyceryloxy, 1,2-O-dialkylglyceryloxy, 1-O-alkyl-2-O-acylglyceryloxy, 1-O-acyl-2-O-alkylglyceryloxy, 1-S-alkyl-2-O-acyl-1-thioglyceryloxy, acyloxymethoxy, S-acyl-2-thioethoxy, S-pivaloyl-2-thioethoxy, acyloxymethoxy, pivaloyloxymethoxy, alkoxycarbonyloxymethoxy, or S-alkyldithio-S′-ethyoxy.

22. The compound according to claim 1 having Formula (XVI): 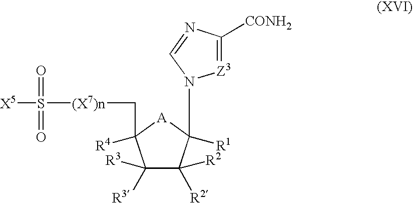
wherein X⁵ is selected from the group consisting of H, F, OH, SH, NH₂, NHOH, N₃, CN, ⁻BH₃M⁺, NHR, R, OR, SR, and R*;
wherein X⁷ is O, S, NH, NMe, CH₂, CHF, CCl₂, or CF₂;
wherein n is 0 or 1; and
wherein Z³ is N, CH, C—OH, or C-ethynyl.

23. The compound according to claim 22 wherein R* is 1,2-O-diacylglyceryloxy, 1,2-O-dialkylglyceryloxy, 1-O-alkyl-2-O-acylglyceryloxy, 1-O-acyl-2-O-alkylglyceryloxy, 1-S-alkyl-2-O-acyl-1-thioglyceryloxy, acyloxymethoxy, S-acyl-2-thioethoxy, S-pivaloyl-2-thioethoxy, acyloxymethoxy, pivaloyloxymethoxy, alkoxycarbonyloxymethoxy, or S-alkyldithio-S′-ethyoxy.

24. The compound according to claim 1 having Formula (XVII): 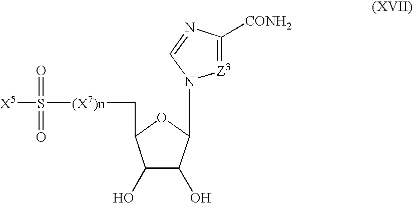
wherein X⁵ is selected from the group consisting of H, F, OH, SH, NH₂, NHOH, N₃, CN, ⁻BH₃M⁺, NHR, R, OR, SR and R*;
wherein X⁷ is O, S, NH, NMe, CH₂, CHF, CCl₂, or CF₂;
wherein n is 0 or 1; and
wherein Z³ is N, CH, C—OH, C-ethynyl.

25. The compound according to claim 24 wherein R* is 1,2-O-diacylglyceryloxy, 1,2-O-dialkylglyceryloxy, 1-O-alkyl-2-O-acylglyceryloxy, 1-O-acyl-2-O-alkylglyceryloxy, 1-S-alkyl-2-O-acyl-1-thioglyceryloxy, acyloxymethoxy, S-acyl-2-thioethoxy, S-pivaloyl-2-thioethoxy, acyloxymethoxy, pivaloyloxymethoxy, alkoxycarbonyloxymethoxy, or S-alkyldithio-S′-ethyoxy.

26. A pharmaceutical composition comprising a therapeutically effective amount of a compound according to claim 1, a pharmaceutically acceptable salt thereof, optionally in combination with one or more other biologically active agents.

27. A method for the treatment of a viral infection comprising administering a therapeutically effective amount of a compound according to claim 1, a pharmaceutically acceptable salt thereof, optionally in combination with one or more antiviral agents.

28. A method for the treatment of a microbial infection comprising administering a therapeutically effective amount of a compound according to claim 1, a pharmaceutically acceptable salt thereof, optionally in combination with one or more antimicrobial agents.

29. A method for the treatment of a proliferative disorder comprising administering a therapeutically effective amount of a compound according to claim 1, a pharmaceutically acceptable salt thereof, optionally in combination with one or more antiproliferative agents.

30. A method for immunomodulation comprising administering a therapeutically effective amount of a compound according to claim 1, a pharmaceutically acceptable salt thereof, optionally in combination with one or more active agents.
